(19) United States
(12) Patent Application Publication
(10) Pub. No.: US 2011/0312985 A1
(43) Pub. Date: Dec. 22, 2011

(54) NARINGENIN COMPLEXES AND METHODS OF USE THEREOF

(75) Inventors: Yaakov Nahmias, Boston, MA (US); Martin L. Yarmush, Newton, MA (US); Raymond T. Chung, Chestnut Hill, MA (US)

(73) Assignee: THE GENERAL HOSPITAL CORPORATION, Boston, MA (US)

(21) Appl. No.: 13/123,357

(22) PCT Filed: Oct. 7, 2009
(86) PCT No.: PCT/US09/59864

§ 371 Date: Sep. 9, 2011

Related U.S. Application Data
(60) Provisional application No. 61/103,701, filed on Oct. 8, 2008.

(51) Int. Cl. A61K 31/505; A61K 31/435; A61P 31/14; A61K 31/366; A61K 31/47; A61K 31/216; A61K 31/40; A61K 31/404

(52) U.S. Cl. 514/275; 514/423; 514/277; 514/419; 514/460; 514/311; 514/510

(57) ABSTRACT

The invention relates to methods of treatment of hepatitis C, dyslipidemia, insulin resistance, and inflammation, with flavonoid-sugar complexes.

### RELATED APPLICATIONS

This application claims the benefit of priority to U.S. Provisional Patent Application Ser. No. 61/103,701, filed Oct. 10, 2008, the contents of which are herein incorporated by reference in their entirety.

### GOVERNMENT SUPPORT

The subject matter of this application was made in part with government support under National Institutes of Health and National Institute of Diabetes and Digestive and Kidney Diseases Grant No. 5 R01 DK043371-13. The U.S. Government has certain rights.

### FIELD OF THE INVENTION

The invention relates to methods of treatment of Hepatitis C, dyslipidemia, insulin resistance, and inflammation.

### BACKGROUND OF THE INVENTION

Hepatitis C virus (HCV) infection is a global public health problem, affecting over 3% of the world population. HCV infection develops into a chronic condition in over 70% of the patients, ultimately leading to cirrhosis and hepatocellular carcinoma (1). Current standards of care consist of interferon (α2A) and ribavirin, which has been found to be effective in only 50% of the cases (1). However, this treatment is poorly tolerated by patients and is associated with significant side effects. Therefore, there is a pressing need for the development of alternative strategies for the treatment of HCV infection.

HCV has long been known to associate with β-lipoproteins (vLDL, LDL) circulating in patients' blood (2). Its receptors E1/E2 were found to bind to both LDL and HDL (3), while HCV core protein was shown to associate with Apolipoprotein AII (Apo AII) (4) and lipid droplets in HepG2 cells(5). In addition, HCV replication has been shown to be upregulated by fatty acids and inhibited by statins, suggesting an interaction between HCV, cholesterol and lipid metabolism (6). The recent development of an efficient cell culture system in which the full lifecycle of HCV infection is captured, opened new opportunities for the study of the viral secretion (7, 8). Using this system, Gastaminza et al. demonstrated that intercellular HCV particles have a higher density than their secreted counterparts, suggesting that HCV might bind low density particles prior to viral egress (9). Just recently, Huang et al. demonstrated that HCV secretion is dependent on both ApoB expression and vLDL assembly in a chromosomally integrated cDNA model of HCV secretion (10). These results strongly suggest that HCV might be ‘hitching a ride’ along the lipoprotein lifecycle. Therefore, compounds previously shown to influence lipoprotein assembly and secretion could possibly exert a similar effect on HCV.

Plant-derived natural products have been used in clinical applications since the dawn of history. In recent years, polyphenols, and flavonoids in particular, have emerged as a class of natural products shown to have antioxidant, antiatherogenic, and normolipidemic effects (78-85). The abundant flavonoid aglycone, naringenin, which is responsible for the bitter taste in grapefruits, has been has been reported to be an antioxidant (87), MTP and ACAT inhibitor (88, 89), and a regulator of cytochrome P450 enzymes including, Cyp1A, 3A4, and 4A10 (90). The ability of naringenin, and its metabolites, to significantly reduce plasma cholesterol levels has been demonstrated both in vivo and in vitro (84, 89, 91). More recently, Huff and coworkers have shown that naringenin helps correct many of the disturbances associated with diabetes in transgenic mice lacking the LDL receptor that were fed a Western-style diet, including correction of VLDL overproduction, amelioration of hepatic steatosis, and attenuation of dyslipidemia (84). Naringenin's clinical relevance is hindered, however, by low solubility and bioavailability owing in part to its largely hydrophobic ring structure.

The present invention is directed to overcoming these deficiencies in the art.

### SUMMARY OF THE INVENTION

In one aspect the invention relates to methods of treating viral infections. The methods include selecting a patient in need of treatment for viral infection and administering to the patient an effective amount of a flavonoid-sugar complex.

In another aspect the invention relates to methods of treating dyslipidemia. The methods include selecting a patient in need of treatment for dyslipidemia and administering to the patient an effective amount of a flavonoid-sugar complex.

In still another aspect the invention relates to methods of treating insulin resistance or diabetes. The methods include selecting a patient in need of treatment for insulin resistance or diabetes and administering to the patient an effective amount of a flavonoid-sugar complex.

In yet still another aspect the invention relates to methods of treating inflammation. The methods include selecting a patient in need of treatment for inflammation and administering to the patient an effective amount of a flavonoid-sugar complex.

### BRIEF DESCRIPTION OF THE DRAWINGS

FIGS. 1A-C show (A) Co-immunoprecipitation (Co-IP) of HCV core protein with Huh7.5.1-secreted ApoB-100. (B) Cell culture secretion of ApoB, HCV positive strand RNA, and HCV core protein in JFH-1 infected Huh7.5.1 cells in response to oleate, insulin, and Brefeldin A. (C) Infectivity of cell culture supernatant assessed by colony formation on naïve Huh7.5.1 cells. (** P<0.01)

FIG. 2 Shows relative secretion of ApoB, HCV positive strand RNA, and HCV core protein in JFH-1 infected Huh7.5.1 cells following silencing of ApoB100 mRNA by SureSilencing shRNA transfection. (** P<0.01)

FIGS. 3A-D show (A) Inhibition of ApoB, HCV positive strand RNA, and HCV core protein secretion by the grapefruit flavonoid Naringenin. (** P<0.01). B) Naringenin inhibits the activity of MTP in a dose-dependent manner. (C) Naringenin induces changes in hepatic gene transcription measured by qPCR. (** P<0.02) (D) Naringenin dose-dependently induces the transcription of PPARα and inhibits the transcription of LXRα as well as HMGR in human heaptocytes.

FIGS. 4A-B show (A) Naringenin stimulation inhibits ApoB secretion of primary human hepatocytes in a dose-dependent manner. (B) Viability of freshly isolated human hepatocytes exposed to increasing concentrations of naringenin for 24 hours.

FIGS. 5A-B show animal survival and liver enzyme release following intraperitoneal (i.p) injection of naringenin to 8 weeks old male SCID mice. (A) Animal survival and liver enzymes. (B) Total triglycerides in animal plasma 24 hours following injection.

FIGS. 6A-B show long term inhibition of HCV RNA secretion by naringenin. (A) Naringenin and IFNα similarly inhibit the secretion of HCV during daily treatment. Naringenin's effect is transient. (B) Intracellular levels of HCV RNA remained unchanged during long-term naringenin treatment.

FIGS. 7A-C show graphs showing UV absorbance measurements of naringenin in the presence of various cyclodextrins. (A) Naringenin UV absorbance with and without β-cyclodextrin. (B) Standard curve of naringenin-β-cyclodextrin complex. (C) Solubility of naringenin in the presence of β-cyclodextrin, Methyl-β-cyclodextrin (MβCD), and Hydroxypropyl-β-cyclodextrin (HPβCD).

FIG. 8 is a graph showing transport of naringenin across a caco-2 cell model intestinal barrier in the presence and absence of Hydroxypropyl-β-cyclodextrin (HPβCD). Lucifer yellow was used as a negative control to measure monolayer permeability during and following the experiment.

FIGS. 9A-B show (A) Results from Gastaminza et al. demonstrating intracellular HCV particles of higher density than their secreted counterparts. (B) Demonstration by Huang et al. of MTP inhibitor blocking HCV secretion. Just recently Gastaminza et al. demonstrated that MTP is essential for the assembly of these high density HCV precursors.

FIG. 10 is a graph showing that Naringenin given by an intra-peritoneal (i.p.) or peroral (p.o.) injection significantly inhibits the growth of murine S-180 tumor implanted in mice

FIG. 11 is a graph showing enhanced oral bioavailability of naringenin with HPβCD in rats.

### DETAILED DESCRIPTION

In one aspect the invention relates to methods of treating viral infections. The methods include selecting a patient in need of treatment for viral infection and administering to the patient an effective amount of a flavonoid-sugar complex.

In some embodiments of this and other aspects described herein, the sugar is a cyclodextrin. In a preferred embodiment, the cyclodextrin is β-cyclodextrin. In a most preferred embodiment, the cyclodextrin is hydroxypropyl-β-cyclodextrin.

In some embodiments of this and other aspects described herein, the flavonoid can be a citrus flavonoid. In some embodiments, the flavonoid can be a flavonone, flavonol, or isoflavone. In preferred embodiments, the flavonone is naringenin

In some embodiments of this and other aspects described herein, the flavonoid-sugar complex is formulated in a pharmaceutically acceptable formulation comprising a pharmaceutically acceptable carrier.

In some embodiments of this and other aspects described herein, the administering is orally administering to the patient in oral dosage form.

In one embodiment of this and other aspects described herein, the oral dosage form is a tablet. In one embodiment of this and other aspects described herein, the tablet is a controlled release tablet.

In one embodiment of this and other aspects described herein, the administering step is from 0 to 4 hours before the patient's next intake of food. In another embodiment of this and other aspects described herein, the administering step is from 1 to 3 hours before the patient's next intake of food. In another embodiment of this and other aspects described herein, the administering step is at least 1 hour before the patient's next intake of food.

In one embodiment of this and other aspects described herein, the administering step is prior to the patients sleep period.

In one embodiment of this and other aspects described herein, the oral dosage form contains from 70 to 5000 mg/dose naringenin. In a preferred embodiment, the dose is from 100 to 1500 mg naringenin for a 70 kg patient. In a more preferred embodiment, the dose is from 250 to 1100 mg naringenin for a 70 kg patient.

Exemplary viral infections that are included in the invention for treatment, include Central European encephalitis virus, Chikungunya virus, Congo-Crimean hemorrhagic fever virus, Dengue viruses 1-4, Eastern equine encephalitis virus, Echoviruses 1-9 and 11-27 and 29-34, Enteroviruses 68-71, Epstein-Barr virus (human herpesvirus 4), Hantaan virus, human Hepatitis A virus, human Hepatitis B virus, human Hepatitis C virus, human herpes simplex viruses 1 and 2, human enteric coronavirus, human cytomegalovirus (human herpesvirus 5), human herpesviruses 6A, 6B, and 7, human immunodeficiency viruses 1 and 2, human respiratory coronaviruses 229E and OC43, human T-lymphotropic viruses 1 and 2, HTLV/BLV viruses, influenza viruses A and B, Japanese encephalitis virus, Kyasanur forest virus, La Crosse virus, Lassa virus, Mayaro virus, Measles virus, Mumps virus, Murray Valley encephalitis virus, Norwalk and related viruses, O'nyong-nyong virus, Omsk hemorrhagic fever virus, Oropouche virus, Papillomaviruses 1-60, Parainfluenza viruses 1, 2, 3 or 4, Parvoviruses, Parvovirus B-19, Polioviruses 1, 2 or 3, RA-1 virus, Picornavirus genus viruses, Rabies virus, Respiratory syncytial virus, Rhinoviruses 1-113, Rift Valley fever virus, Rocio virus, Ross River virus, Rubella virus, Russian spring-summer encephalitis virus, Sandfly fever-Naples virus, Sandfly fever-Sicilian virus, St. Louis encephalitis virus, SV 40 virus, Tahyna virus, Vaccinia virus, Varicella-zoster virus (human herpesvirus 3), Variola virus, Venezuelan equine encephalitis virus, Vesicular stomatitis viruses, West Nile virus, Eastern equine encephalitis virus, Yellow fever virus, Avian reticuloendotheliosis virus, Avian sarcoma and leukosis viruses, B virus (Cercopithecus herpesvirus), Berne virus (horses), Border disease virus (sheep), Bovine enteroviruses 1-7, Bovine ephemeral fever virus, Bovine immunodeficiency virus, Bovine leukemia virus, Bovine mamillitis virus, Bovine papillomaviruses, Bovine papular stomatitis virus, Bovine respiratory syncytial virus, Bovine virus diarrhea virus, Breda virus (calves), Canine adenovirus 2, Canine distemper virus, Canine parvovirus, Caprine arthritis-encephalitis virus, Eastern equine encephalitis virus, Encephalomyocarditis virus, Equine abortion virus, Equine adenoviruses, Equine coital exanthema virus, Equine infectious anemia virus, Equine rhinopneumonitis virus (EHV4), Feline immunodeficiency virus, Feline infectious peritonitis virus, Feline panleukopenia virus, Feline sarcoma and leukemia viruses, Foot-and-mouth disease viruses, Hemagglutinating encephalomyelitis virus (swine), Hog cholera virus, Infectious bovine rhinotracheitis virus, Infectious bronchitis virus (fowl), Infectious canine hepatitis virus, Infectious hematopoietic necrosis virus (fish), Infectious laryngotracheitis virus (fowl), Influenza viruses of swine, horses, seals, and fowl, Japanese encephalitis virus, Maedilvisna virus (sheep), Marek's disease virus (fowl), Mink enteritis virus, Minute virus of mice, Mouse hepatitis viruses, Mouse mammary tumor virus, Mouse poliomyelitis virus (Theiler's virus), Mucosal disease virus (cattle), Newcastle disease virus (fowl), Parainfluenza virus 3, Parainfluenza virus 1 (Sendai virus), Peste-des-petits-ruminants virus (sheep and goats), Pneumonia virus of mice, Progressive pneumonia virus of sheep, Pseudorabies virus, Rabies virus, Rift Valley fever virus, Rinderpest virus, Rotaviruses, Shope papillomavirus, Simian immunodeficiency viruses (SIV, SHIV), Swine rubivirus is rubella.vesicular disease virus, Tick-borne encephalitis viruses, Transmissible gastroenteritis virus (swine), Turkey blue-comb virus, Venezuelan equine encephalitis virus, Vesicular stomatitis viruses, Wesselsbron virus and Western equine encephalitis virus. In one preferred embodiment, the viral infection is a hepatitis C virus infection.

The interaction between HCV infection, cholesterol and fatty acid metabolism has received significant attention mainly due to the development of liver steatosis in chronically infected patients (19). However, the lack of an efficient cell culture model of HCV replication and infection has significantly limited research in the field. Despite these limitations, several groups demonstrated that HCV core protein associates with ApoAII (4) and lipid droplets in HepG2 cells (5) over expressing the protein. E1/E2 proteins of HCV has been shown to bind to both LDL and HDL (3), The data suggested that HCV in infected patients might circulate as lipo-viral particles (LVP) (20). The development of HCV replicon system (21) allowed for the efficient study of viral replication in culture. Using this system Kapadia and Chisari demonstrated that HCV replication is regulated by geranylgeranylation and fatty acid metabolism (6). Others demonstrated that HCV nonstructural proteins, such as NS5A, inhibit ApoB secretion (22).

The recent development of the JFH-1 virus (7) in combination with the Huh-7.5.1 cell-line (8) allowed for the efficient infection of cells and the generation of large virus titers in culture. This model allowed for the identification of intercellular infectious HCV particles with higher density than their secreted counterparts(9) suggesting the binding of HCV to low density particles in the ER. Just recently, Huang et al. demonstrated that HCV assembled in ApoB and MTP enriched vesicles, and that the viral secretion was dependent on both ApoB expression and vLDL assembly in a chromosomally integrated cDNA model of HCV secretion (10). As the association between HCV and serum β-lipoproteins (vLDL, LDL) is well known (2), these results strongly suggest that HCV might ‘hitch a ride’ on the lipoprotein-cholesterol lifecycle. This hypothesis is intriguing as it might explain the presence of HCV in intestinal cells, a second site of lipoprotein production (23). In addition, it might explain HCV uptake by LDL-R (24, 25), SR-BI (26), and heparin sulfate (27).

Our results strongly support this hypothesis. We demonstrate that HCV produced by the Huh7.5.1 cell line is bound to ApoB, and that its secretion is inhibited by brefeldin A, a metabolite of the fungus Eupenicillium brefeldianum, which blocks the communication between the endoplasmic reticulum and the Golgi, effectively inhibiting protein secretion (12, 13). We also demonstrate that HCV secretion is upregulated by the fatty acid oleate and downregulated by insulin, precisely minoring ApoB secretion by the cells(12). Moreover, silencing ApoB100 mRNA caused a significant and parallel decrease in HCV core protein secretion. In addition to demonstrating the relationship between HCV and vLDL secretion in culture, our results also suggest a novel therapeutic approach for the treatment of HCV infection.

### The HCV Life Cycle

HCV is an enveloped, ˜9500 bp, positive-strand RNA virus, a member of the Flaviviridae family. The viral genome encodes a single open reading frame of approximately 3000 amino acids. The viral life cycle begins upon entry into the host cell. The process of cellular entry has yet to be clarified completely, but upon introduction of the viral genetic material into the host cytoplasm, translation is initiated via the viral 5′ non-translated region, which functions as a ribosomal entry site (44). The viral polyprotein is threaded in and out of the endoplasmic reticulum (ER), and is then cleaved both by host enzymes and autocatalytically by proteases that are part of the nascent polyprotein. This leads to the production of mature structural and nonstructural (NS) proteins (45). The accumulation of viral proteins in the cellular ER induces morphological changes in the cell with the formation of a membranous web, where viral replication has been reported to occur (46). The viral NS5B protein is the RNA-dependent RNA polymerase, which in collaboration with other viral proteins and virally-induced structures in the cells replicates the viral RNA genome via a negative strand intermediate (47). The HCV virus is then thought to be secreted into the ER as a high density particle which associates with nascent vLDL particles (42, 48). Our group recently demonstrated that HCV is then activity secreted in a Golgi-dependent mechanism while bound to vLDL. Persistent HCV infection is thought to be dependent on high viral titers causing repeated incidents of hepatocyte infection and subsequent clearance(49). Therefore treatments aimed at reducing the circulating viral titers would allow non-infected liver cells to regenerate and replace dying cells which replicate the virus.

HCV Interaction with Cholesterol and Fatty Acid Metabolism

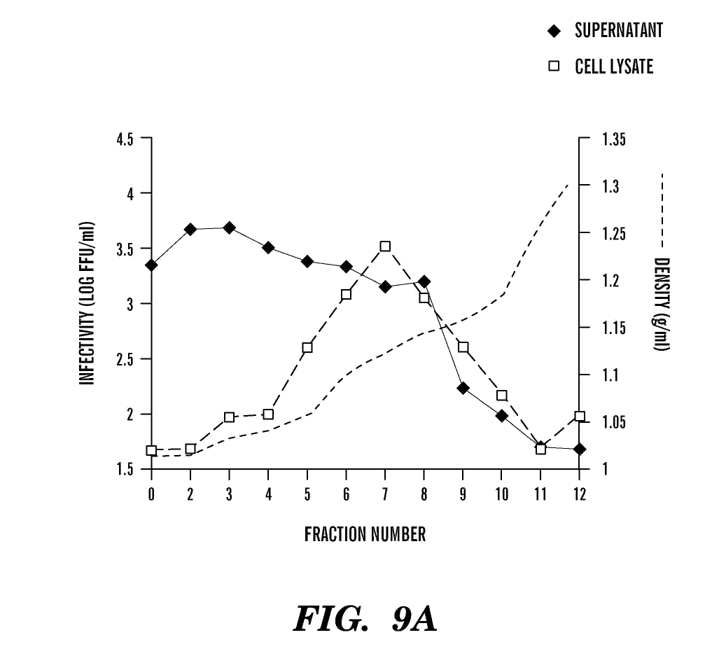

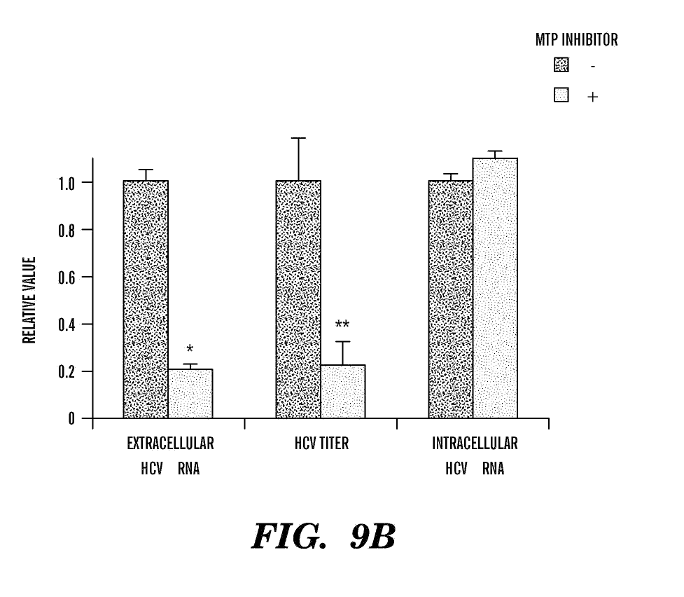

The interaction between HCV infection, cholesterol and fatty acid metabolism has received significant attention mainly due to the development of liver steatosis in chronically infected patients (40, 50). However, the lack of an efficient cell culture model of HCV has significantly limited research in the field. In spite of these limitations, several groups demonstrated that HCV core protein associates with Apolipoprotein AII (51) and lipid droplets in HepG2 cells over expressing the HCV core protein (52, 53). The development of HCV replicon system (54) allowed for the efficient study of viral replication in culture. Using this system, our group and others, have shown that HCV replication is inhibited by statins and enhanced by the additions of fatty acids (41, 55). This was shown to be in part due to the viral requirement for geranylgeranylation (41). Just recently, Gastaminza et al. demonstrated the existence of high density intracellular HCV precursors suggesting the virus binds to low density particles in the ER (48). Using a similar system Huang et al. demonstrated that HCV assembled in ApoB and MTP enriched vesicles (42) while our group and others demonstrated that HCV is actively secreted while bound to vLDL (43, 56). As the association between HCV and serum lipoproteins is well known (57), these results strongly suggest that HCV ‘hitches a ride’ on the lipoprotein lifecycle. This hypothesis explains HCV uptake by receptors involved in lipoprotein uptake such as the LDL-R (58, 59), SR-BI (60), and heparin sulfate (61). See FIG. 9.

### Grapefruit Flavonoid Naringenin

Naringin, is an abundant flavonoid found in citrus fruits responsible to the bitter taste in grapefruit. Naringin is hydrolyzed by enterobacteria to naringenin prior to being absorbed by the intestine. Naringenin has been reported to be an antioxidant (62), MTP and ACAT inhibitor(63), and a regulator of cytochrome P4503A and 4A activity (64, 65). The ability of naringenin, or its glycosylated form, to significantly reduce plasma cholesterol levels has been demonstrated both in vivo and in vitro (66, 67). A recent clinical trial in hypercholesterolemic patients demonstrated that a low dose of naringin (400 mg/day) lowered LDL levels by 17% (68). Similar cholesterol lowering effect of naringenin were demonstrated in rabbit (66, 69) and rats (70). The concept of supplementing the diet of HCV patient's with naringenin is appealing as the compound is simple, cheap, stable, and readily available as a food additive.

### Naringenin's Antioxidant, Anti-Carcinogenic Properties

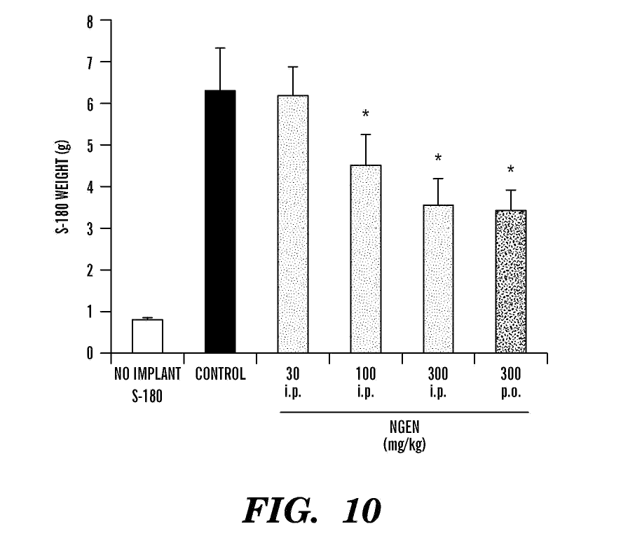

Flavonoids, such as naringenin, have been ascribed with anti-carcinogenic properties. The compounds were demonstrated to cause apoptosis in a variety of tumor cell lines, including human hepatoma cell lines HepG2 and Huh7 (62). This activity is thought to be mediated by the activation of phase II enzymes, such as glutathione S-transferase, which results in the detoxification of carcinogens (64). Naringenin was shown to induce glutathione S-transferases in mice by 4 to 8 folds (71). It was also shown to increase hepatic superoxide dismutase and glutathione peroxidase activities in rats (72). Finally, naringenin was demonstrated to significantly inhibit the tumorgrowth of S-180 sarcoma cell line, implanted in mice, following intraperitoneal or peroral injection once a day for 5 days (62). See FIG. 10.

### Naringenin's Hypolipidemic Properties

The hypolipidemic effects of citrus flavonoids have been studied extensively both in vitro and in vivo. In a fairly recent clinical trial, a low oral dose of naringenin (400 mg/day) was shown to reduce circulating LDL levels by 17% in a group of 30 hypercholesterolemic patients (68). Much of the molecular mechanism was studied in HepG2 cells, where naringenin and hesperetin were shown to reduce the secretion of ApoB-containing lipoproteins and suppress cellular synthesis of cholesteryl esters and triglycerides (63, 73). In that model naringenin has been shown to inhibit ACAT2 (63), an enzyme responsible for the synthesize of cholesteryl esters, as well as downregulate MTP (74, 75), an enzyme which catalyses the transfer of lipids, primarily triglycerides, to nascent ApoB in the ER. Allister et al. demonstrated that this inhibition is regulated primarily through the mitogen-activated protein kinase (MAPK^{erk}) pathway, through the activation of MEK1/2 and ERK1/2 (67). In addition, both naringenin and hespertin were shown to increase the expression of the LDL receptor (LDL-R) which is responsible for lipoprotein clearance (63). This upregulation of LDL-R was shown to be caused by activation of phosphatidylinositol 3-kinase (PI3K) upstream of SREBP-1 (75). Interestingly, naringenin was also shown to inhibit HMGR while activating enzymes important in fatty acid oxidation such as acyl-coenzyme A oxidase (Aox), and cytochrome P450 IV A1 (65). The myriad effects induced by naringenin suggest that the flavonoid target might be at the nuclear receptor level. Strengthening this hypothesis is the report that naringenin binds to the liver x receptor (LXR) (75) while the closely related polymethoxyflavone, tangeretin, activates the peroxisome proliferator-activated receptor α (PPARα)(73). Such genetic control of metabolism was demonstrated for green tea polyphenols which downregulate LXRα while upregulating PPARα as well as for soy isoflavones(76, 77).

HCV infection is a global public health problem, affecting 3% of the world population. The recent development of HCV replicon system followed by the JFH1/Huh7.5.1 full lifecycle model of HCV infection allowed for the study of HCV infection in culture. These studies demonstrated a direct interaction between HCV lifecycle to cholesterol and fatty acid metabolism. Recent work, suggests HCV replication is dependent on geranylgeranylation and inhibited by statins (41), while HCV egress is dependent on vLDL assembly and secretion (42, 43). The grapefruit flavonoid naringenin is a non-toxic antioxidant with demonstrated anti-inflammatory and anti-carcinogenic properties. In vitro and in vivo studies demonstrated that naringenin inhibits vLDL secretion through multiple mechanisms, suggesting an underlying transcriptional regulation.

Naringin, one of the most abundant flavonoids in citrus fruits, is hydrolyzed by enterobacteria to naringenin prior to being absorbed. Naringenin has been reported to be an antioxidant (28), MTP and ACAT inhibitor(16), and a regulator of cytochrome P4503A and 4A activity (29, 30). The ability of naringenin, or its glycosylated form, to significantly reduce plasma cholesterol levels has been demonstrated both in vivo and in vitro (14, 15). It is thought that naringenin inhibits the expression and activity of MTP, which catalyzes the transfer of lipids to the nascent ApoB molecule as it buds into the endoplasmic reticulum as a vLDL particle (16-18). Our results demonstrate that short-term (24 hrs) stimulation of infected hepatocytes with 200 μM naringenin significantly inhibits HCV secretion by 80%±10% and the infectivity of the titer by 79%±10%. At the same time, transcription of the viral RNA remains unchanged. We suggest that this is due in part to the inhibition of MTP activity by 58%±8% as well as the inhibition of HMGR and ACAT2 transcription. Long-term (3 days) stimulation with naringenin had an even greater effect, inhibiting HCV secretion by 96±5% comparable to the effects of the current standard-of-care, 1000 i.u. of Interferon α (IFNα), which inhibited HCV secretion by 93±5%. To further demonstrate naringenin as a potential therapy we show the compound is non-toxic to freshly isolated human hepatocytes up to concentrations greater than 1000 μM. In addition, we demonstrate that naringenin induced a 60%±7% decrease in ApoB secretion by primary human hepatocytes.

The concept of supplementing HCV patient's diets with naringenin is appealing. A recent clinical trial in hypercholesterolemic patients demonstrated that a low dose of naringin (400 mg/day) lowered LDL levels by 17% (31). Similar cholesterol-lowering effect of naringenin were demonstrated in rabbits (14, 32) and rats (33). However, it is worth noting that the absorbance of naringenin through the intestinal wall is extremely limited (about 6%) necessitating the development of an oral delivery platform which could transverse the intestinal barrier. Prior studies suggested that the LD50 (50% kill) for naringenin is 2000 mg/kg for both rats and guinea pigs by intraperitoneal injection (34). Our results show that doses up to 1500 mg/kg naringenin given by intraperitoneal injection to mice did not cause death or a marked elevation of liver enzymes suggesting naringenin does not display hepatic toxicity even following intravenous administration.

The ability of the liver to regenerate as well as the RNA-based lifecycle of HCV allow for a potential clearance of the viral infection. It is thought that clearance occurs in about 30% of HCV infected patients. The possible reduction of HCV viral load by inhibiting viral secretion could allow uninfected cells to regenerate, potentially increasing the overall rate of viral clearance.

Thus in one aspect, the invention provides methods of reducing viral load. The methods include selecting a patient in need of treatment for viral infection and administering to the patient an effective amount of a compound that inhibits virus secretion. In some embodiments, the compound that inhibits virus section is a flavonoid or a flavonoid-sugar complex.

One benefit of reduced viral load can better clearance of virus by antiviral compounds. For example, a composition of the invention can be given together with an antiviral compound. Therefore, in some embodiments of this and other aspects described herein, the flavonoid-sugar complex is administrated together with an antiviral compound. Many antiviral compounds are known in the art and easily available to one of skill in the art. One exemplary antiviral compound is interferon alpha.

In some embodiments of this and other aspects described herein, the sugar is a cyclodextrin. In a preferred embodiment, the cyclodextrin is β-cyclodextrin. In a most preferred embodiment, the cyclodextrin is hydroxypropyl-β-cyclodextrin.

In some embodiments of this and other aspects described herein, the flavonoid can be a citrus flavonoid. In some embodiments, the flavonoid can be a flavonone. In preferred embodiments, the flavonone is naringenin.

In some embodiments of this and other aspects described herein, the flavonoid-sugar complex is formulated in a pharmaceutically acceptable formulation comprising a pharmaceutically acceptable carrier.

In some embodiments of this and other aspects described herein, the administering is orally administering to the patient in oral dosage form.

In one embodiment of this and other aspects described herein, the oral dosage form is a tablet. In one embodiment of this and other aspects described herein, the tablet is a controlled release tablet.

In another embodiment of this and other aspects described herein, the administering step is from 0 to 4 hours before the patient's next intake of food. In another embodiment of this and other aspects described herein, the administering step is from 1 to 3 hours before the patient's next intake of food. In another embodiment of this and other aspects described herein, the administering step is at least 1 hour before the patient's next intake of food.

In another embodiment of this and other aspects described herein, the administering step is prior to the patients sleep period.

In one embodiment of this and other aspects described herein, the oral dosage form contains from 70 to 5000 mg/dose naringenin. In a preferred embodiment, the dose is from 100 to 1500 mg naringenin for a 70 kg patient. In a more preferred embodiment, the dose is from 250 to 1100 mg naringenin for a 70 kg patient.

In another aspect, the invention provides methods for inhibiting the secretion of a virus from a cell, the method comprising contacting a cell with a flavonoid or a flavonoid-sugar complex.

In still another aspect the invention relates to methods of treating dyslipidemia. The methods include selecting a patient in need of treatment for dyslipidemia and administering to the patient an effective amount of a flavonoid-sugar complex.

In some embodiments of this and other aspects described herein, the sugar is a cyclodextrin. In a preferred embodiment, the cyclodextrin is β-cyclodextrin. In a most preferred embodiment, the cyclodextrin is hydroxypropyl-β-cyclodextrin.

In some embodiments of this and other aspects described herein, the flavonoid can be a citrus flavonoid. In some embodiments, the flavonoid can be a flavonone. In preferred embodiments, the flavonone is naringenin.

In some embodiments of this and other aspects described herein, the flavonoid-sugar complex is formulated in a pharmaceutically acceptable formulation comprising a pharmaceutically acceptable carrier.

In some embodiments of this and other aspects described herein, the administering is orally administering to the patient in oral dosage form.

In one embodiment of this and other aspects described herein, the oral dosage form is a tablet. In one embodiment of this and other aspects described herein, the tablet is a controlled release tablet.

In another embodiment of this and other aspects described herein, the administering step is from 0 to 4 hours before the patient's next intake of food. In another embodiment of this and other aspects described herein, the administering step is from 1 to 3 hours before the patient's next intake of food. In another embodiment of this and other aspects described herein, the administering step is at least 1 hour before the patient's next intake of food.

In another embodiment of this and other aspects described herein, the administering step is prior to the patients sleep period.

In one embodiment of this and other aspects described herein, the oral dosage form contains from 70 to 5000 mg/dose naringenin. In a preferred embodiment, the dose is from 100 to 1500 mg naringenin for a 70 kg patient. In a more preferred embodiment, the dose is from 250 to 1100 mg naringenin for a 70 kg patient.

In still another aspect the invention relates to methods of treating insulin resistance or diabetes. The methods include selecting a patient in need of treatment for insulin resistance or diabetes and administering to the patient an effective amount of a flavonoid-sugar complex.

In some embodiments of this and other aspects described herein, the sugar is a cyclodextrin. In a preferred embodiment, the cyclodextrin is β-cyclodextrin. In a most preferred embodiment, the cyclodextrin is hydroxypropyl-β-cyclodextrin.

In some embodiments of this and other aspects described herein, the flavonoid can be a citrus flavonoid. In some embodiments, the flavonoid can be a flavonone. In preferred embodiments, the flavonone is naringenin.

In some embodiments of this and other aspects described herein, the flavonoid-sugar complex is formulated in a pharmaceutically acceptable formulation comprising a pharmaceutically acceptable carrier.

In some embodiments of this and other aspects described herein, the administering is orally administering to the patient in oral dosage form.

In one embodiment of this and other aspects described herein, the oral dosage form is a tablet. In one embodiment of this and other aspects described herein, the tablet is a controlled release tablet.

In another embodiment of this and other aspects described herein, the administering step is from 0 to 4 hours before the patient's next intake of food. In another embodiment of this and other aspects described herein, the administering step is from 1 to 3 hours before the patient's next intake of food. In another embodiment of this and other aspects described herein, the administering step is at least 1 hour before the patient's next intake of food.

In another embodiment of this and other aspects described herein, the administering step is prior to the patients sleep period.

In one embodiment of this and other aspects described herein, the oral dosage form contains from 70 to 5000 mg/dose naringenin. In a preferred embodiment, the dose is from 100 to 1500 mg naringenin for a 70 kg patient. In a more preferred embodiment, the dose is from 250 to 1100 mg naringenin for a 70 kg patient.

In yet still another aspect the invention relates to methods of treating inflammation. The methods include selecting a patient in need of treatment for inflammation and administering to the patient an effective amount of a flavonoid-sugar complex.

In some embodiments of this and other aspects described herein, the sugar is a cyclodextrin. In a preferred embodiment, the cyclodextrin is β-cyclodextrin. In a most preferred embodiment, the cyclodextrin is hydroxypropyl-β-cyclodextrin.

In some embodiments of this and other aspects described herein, the flavonoid can be a citrus flavonoid. In some embodiments, the flavonoid can be a flavonone. In preferred embodiments, the flavonone is naringenin.

In some embodiments of this and other aspects described herein, the flavonoid-sugar complex is formulated in a pharmaceutically acceptable formulation comprising a pharmaceutically acceptable carrier.

In some embodiments of this and other aspects described herein, the administering is orally administering to the patient in oral dosage form.

In one embodiment of this and other aspects described herein, the oral dosage form is a tablet. In one embodiment of this and other aspects described herein, the tablet is a controlled release tablet.

In another embodiment of this and other aspects described herein, the administering step is from 0 to 4 hours before the patient's next intake of food. In another embodiment of this and other aspects described herein, the administering step is from 1 to 3 hours before the patient's next intake of food. In another embodiment of this and other aspects described herein, the administering step is at least 1 hour before the patient's next intake of food.

In another embodiment of this and other aspects described herein, the administering step is prior to the patients sleep period.

In one embodiment of this and other aspects described herein, the oral dosage form contains from 70 to 5000 mg/dose naringenin. In a preferred embodiment, the dose is from 100 to 1500 mg naringenin for a 70 kg patient. In a more preferred embodiment, the dose is from 250 to 1100 mg naringenin for a 70 kg patient.

In one more aspect the invention provides methods for inhibiting LXR in a cell. the method comprising contacting a cell with a flavonoid or a flavonoid-sugar complex. This aspect of the invention can be used to treat patients in need of treatment for a disease where inhibiting LXR can be beneficial. [[Any known disease?]] The method can include selecting a patient in need of treatment for a disease where inhibiting LXR can be beneficial and administering to the patient an effective amount of a flavonoid-sugar complex.

As used herein, the term “cyclodextrin” is intended to mean a cyclodextrin or a derivative thereof as well as mixtures of various cyclodextrins, mixtures of various derivatives of cyclodextrins and mixtures of various cyclodextrins and their derivatives. The cyclodextrin may be selected from the group consisting of alpha-cyclodextrin, beta-cyclodextrin, gamma-cyclodextrin and derivatives thereof. The cyclodextrin may be modified such that some or all of the primary or secondary hydroxyl groups of the macrocycle are alkylated or acylated. Methods of modifying these hydroxyl groups are well known to the person skilled in the art and many such modified cyclodextrins are commercially available. Thus, some or all of the hydroxyl groups of the cyclodextrin may have been substituted with an O—R group or an O—C(O)—R group, wherein R is an optionally substituted C₁-C₆ alkyl, an optionally substituted C₂-C₆ alkenyl, an optionally substituted C₂-C₆ alkynyl, an optionally substituted aryl or heteroaryl group. Thus, R may be a methyl, an ethyl, a propyl, a butyl, a pentyl, or a hexyl group, i.e. O—C(O)—R may be an acetate. Furthermore, the hydroxyl groups may be per-benzylated, per-benzoylated, benzylated or benzoylated on just one face of the macrocycle, i.e. only 1, 2, 3, 4, 5 or 6 hydroxyl groups is/are benzylated or benzoylated. Naturally, the hydroxyl groups may also be per-alkylated or per-acylated, such as per-methylated or per-acetylated, alkylated or acylated, such as methylated or acetylated, on just one face of the macrocycle, i.e. only 1, 2, 3, 4, 5 or 6 hydroxyl groups is/are alkylated or acylated, such as methylated or acetylated. Preferably cyclodextrin can be a β-cyclodextrin, more preferably hydroxypropyl-β-cyclodextrin. Other cyclodextrins and cyclodextrin derivatives that are amenable to the invention are described in U.S. Pat. Nos. 5,385,891; 5,929,131; 5,241,059;6,045,812; 6,046,177; 5,792,821 6,204,256; 5,910,551; 4,764,604; 5,916,883; 5,728,823; 5,594,125; 5,134,127; and 5,248,675, contents of all of which are herein incorporated by reference for all purposes.

In one aspect, the invention relates to pharmaceutical compositions comprising a flavonoid-sugar complex. In certain embodiments the sugar can be a cyclodextrin. In a preferred embodiment, the cyclodextrin is β-cyclodextrin. In a most preferred embodiment, the cyclodextrin is hydroxypropyl-β-cyclodextrin.

In some embodiments of this and other aspects described herein, the flavonoid can be a citrus flavonoid. In some embodiments, the flavonoid can be a flavonone. In preferred embodiments, the flavonone is naringenin.

The invention also relates to pharmaceutical compositions comprising a naringenin-sugar complex. The invention also relates to pharmaceutical compositions consisting essentially of a naringenin β-cyclodextrin complex.

In one embodiment of this and other aspects described herein, the sugar is a cyclodextrin. In a preferred embodiment, the cyclodextrin is β-cyclodextrin. In a most preferred embodiment, the cyclodextrin is hydroxypropyl-β-cyclodextrin.

As used herein, the term “flavonoid” refers to a class of natural or synthetic plant secondary metabolites based around a phenylbenzopyrone structure and are also commonly referred to by the equivalent term “bioflavonoid”. Flavonoids include, but are not limited to, flavones, flavonols, flavanones, flavan-3-ols, isoflavones, anthocyanadins, and proanthocyanidins. Flavones include, but are not limited to, luteolin and apigenin. Flavonols include, but are not limited to, quercetin, kaempferol, myricetin, isorhamnetin, pachypodol, and rhamnazin. Flavanones include, but are not limited to, hesperetin, naringenin, and eriodictyol. Flavan-3-ols include, but are not limited to, (+)-catechin, (+)-gallocatechin, (−)-epicatechin, (−)-*epigallocatechin, (−)-epicatechin 3-gallate, (−)-epigallocatechin 3-gallate, theaflavin, theaflavin 3-gallate, theaflavin 3′-gallate, theaflavin 3,3′ digallate, and thearubigins. Isoflavones include, but are not limited to, genistein, daidzein, and glycitein. Anthocyanidins include, but are not limited to, cyanidin, delphinidin, malvidin, pelargonidin, peonidin, and petunidin. Some Exemplary flavonoids are described in U.S. Pat. Nos. 6,028,088 and 6,28,042; and in U.S. Pat. Publication No. 2007/0254859, contents of all of which are herein incorporated by reference for all purposes.

As used herein, the term “treatment” or “treating” includes preventing, lowering, stopping, or reversing the progression or severity of the condition or symptoms associated with a condition being treated. As such, the term “treatment” or “treating” includes medical, therapeutic, and/or prophylactic administration, as appropriate.

As used herein, the term “inhibit” means complete eradication or partial reduction.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

As used herein, the terms “active agent” or “agent” refers a flavonoid complexed with sugar, e.g., a naringenin-cyclodextrin complex.

Agents can be administered orally, parenterally, for example, subcutaneously, intravenously, intramuscularly, intraperitoneally, by intranasal instillation, or by application to mucous membranes, such as, that of the nose, throat, and bronchial tubes. They can be administered alone or with suitable pharmaceutical carriers, and can be in solid or liquid form such as, tablets, capsules, powders, solutions, suspensions, or emulsions.

The active agent can be formulated in pharmaceutically acceptable compositions which comprise a therapeutically-effective amount of the active agent, formulated together with one or more pharmaceutically acceptable carriers (additives) and/or diluents. The active agents can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. Additionally, active agents can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

“PEG” means an ethylene glycol polymer that contains about 20 to about 2000000 linked monomers, typically about 50-1000 linked monomers, usually about 100-300. Polyethylene glycols include PEGs containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

The active agents can be formulated in a gelatin capsule, in tablet form, dragee, syrup, suspension, topical cream, suppository, injectable solution, or kits for the preparation of syrups, suspension, topical cream, suppository or injectable solution just prior to use. Also, active agents can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17”” edition, Nema et al., PDA J. Pharm. Sci. Tech. 1997 51:166-171. Methods to make invention formulations include the step of bringing into association or contacting an ActRIIB active agent with one or more excipients or carriers. In general, the formulations are prepared by uniformly and intimately bringing into association one or more active agents with liquid excipients or finely divided solid excipients or both, and then, if appropriate, shaping the product.

The preparative procedure may include the sterilization of the pharmaceutical preparations. The compounds may be mixed with auxiliary agents such as lubricants, preservatives, stabilizers, salts for influencing osmotic pressure, etc., which do not react deleteriously with the compounds.

Examples of injectable form include solutions, suspensions and emulsions. Injectable forms also include sterile powders for extemporaneous preparation of injectible solutions, suspensions or emulsions. The compounds of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatinA suitable carrier is 5% dextrose in saline. Frequently, it is desirable to include additives in the carrier such as buffers and preservatives or other substances to enhance isotonicity and chemical stability.

In some embodiments, active agents can be administrated encapsulated within liposomes. The manufacture of such liposomes and insertion of molecules into such liposomes being well known in the art.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and Primogel™, and the like.

The active agents can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active agents may be incorporated with excipients and used in the form of tablets, capsules, elixirs, suspensions, syrups, and the like. Such compositions and preparations should contain at least 0.1% of active agent. The percentage of the agent in these compositions may, of course, be varied and may conveniently be between about 2% to about 60% of the weight of the unit. The amount of active agent in such therapeutically useful compositions is such that a suitable dosage will be obtained. Preferred compositions according to the present invention are prepared so that an oral dosage unit contains between about 100 and 2000 mg of active agent.

Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives.

The compositions can be given as a bolus dose, to maximize the circulating levels for the greatest length of time after the dose. Continuous infusion may also be used after the bolus dose.

The active agents can also be administrated directly to the airways in the for of an aerosol. For administration by inhalation, the active agents in solution or suspension can be delivered in the form of an aerosol spray from pressured container or dispenser which contains a suitable propellant, e.g., a gas such as carbon dioxide, or hydrocarbon propellant like propane, butane or isobutene. The active agents can also be administrated in a no-pressurized form such as in an atomizer or nebulizer.

The active agents can also be administered parenterally. Solutions or suspensions of these active agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of active compound calculated to produce the desired therapeutic effect in association with the required pharmaceutical carrier.

The methods of the invention further include administering to a subject a therapeutically effective amount of an active agent in combination with another pharmaceutically active compound. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference.

The active agent and another pharmaceutically active compound may be administrated to the subject in the same pharmaceutical composition or in different pharmaceutical compositions (at the same time or at different times). In some embodiments, the pharmaceutically active compound is a HMG-CoA reductase inhibitor, e.g. a statin. Exemplary statins include, but are not limited to, atrovastatin (Lipitor, Torvast), cerivastatin (Lipobay, Baycol). Fluvastatin (Lescol, Lescol XL), lovastatin (Mevacor, Altocor, Altoprev), mevastatin, pitavastin (Livalo, Pitava), praystatin (Pravachol, Selektine, Lipostat), rosuvastatin (Crestor), and simvastatin (Zocor, Lipex).

Cytochrome P450 (abbreviated CYP, P450, infrequently CYP450) is a very large and diverse superfamily of hemoproteins found in all domains of life. Cytochromes P450 use a plethora of both exogenous and endogenous compounds as substrates in enzymatic reactions. Usually they form part of multicomponent electron transfer chains, called P450-containing systems.

Human CYPs are primarily membrane-associated protein, located either in the inner membrane of mitochondria or in the endoplasmic reticulum of cells. CYPs metabolize thousands of endogenous and exogenous compounds. Most CYPs can metabolize multiple substrates, and many can catalyze multiple reactions, which accounts for their central importance in metabolizing the extremely large number of endogenous and exogenous molecules. In the liver, these substrates include drugs and toxic compounds as well as metabolic products such as bilirubin (a breakdown product of hemoglobin). Cytochrome P450 enzymes are present in most other tissues of the body, and play important roles in hormone synthesis and breakdown (including estrogen and testosterone synthesis and metabolism), cholesterol synthesis, and vitamin D metabolism. The Human Genome Project has identified 57 human genes coding for the various cytochrome P450 enzymes.

All drugs are detoxified and eventually excreted from the body, and many require bioactivation to form the active compound. CYPs are the major enzymes involved in drug metabolism and bioactivation, accounting for ˜75% of the total metabolism. As used herein, the term “metabolism” refers to chemical modification and/or degradation of pharmaceutically active compounds.

Changes in CYP enzyme activity can affect the metabolism and clearance of various drugs that are metabolized by CYPs. For example, if one drug inhibits the CYP-mediated metabolism of another drug, the second drug can be used at a lower dosage to achieve to higher plasma concentration due to lowered drug metabolism. The effective dose is lowered and/or efficacy is increased, i.e. bioavailability of the second drug is increased.

In one aspect, the invention provides methods for increasing the bioavailability of a pharmaceutically active compound, the method comprising administering to a subject a pharmaceutically active compound (drug) and a flavonoid-sugar complex, wherein the subject is in need of treatment with the pharmaceutically active compound and the pharmaceutically active compound is metabolized by a CYP enzyme. As discussed above, the pharmaceutically active compound and flavonoid-sugar complex may be administrated to the subject in the same pharmaceutical composition or in different pharmaceutical compositions (at the same time or at different times).

The increase in bioavailability can be determined by measuring total systemic drug concentrations over time after coadministration of drug with flavonoid-sugar complex. The increase in drug bioavailability is defined as an increase in Area Under the Curve (AUC). AUC is the integrated measure of systemic drug concentration over time in units of mass-time/volume. The AUC from time zero (time of administration) to time infinity (when no drug remains in the body) following administration of a drug is a measure of the subject to the drug.

Systemic drug concentrations can be measured using standard in vitro or in vivo drug measurement techniques. As used herein, the term “systemic drug concentration: refers to a drug concentration in a subject's bodily fluids, such as serum, plasma, and/or blood; the term also includes drug concentrations in tissues bathed by the systemic fluids, including the skin. The increase in total systemic drug concentration is one way of defining an increase in drug bioavailability.

In some embodiments, the pharmaceutically active compound is a HMG-CoA reductase inhibitor, e.g. a statin.

In one embodiment of this and other aspects described herein, the flavonoid-sugar complex is administrated orally to the patient in an oral dosage form.

In one embodiment of this and other aspects described herein, the oral dosage form contains from 1 to 5000 mg/dose naringenin.

In one embodiment of this and other aspects described herein, the oral dosage form is a tablet.

In one embodiment of this and other aspects described herein, the tablet is a controlled release tablet.

In one embodiment of this and other aspects described herein, the sugar is hydrpxypropyl-β-cyclodextrin.

In one embodiment of this and other aspects described herein, the flavonoid is naringenin.

The amount of active agent which can be combined with a carrier material to produce a single dosage form will generally be that amount of the compound which produces a therapeutic effect. Generally out of one hundred percent, this amount will range from about 0.1% to 99% of active agent, preferably from about 5% to about 70%, most preferably from 10% to about 30%.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

As used herein, the term “therapeutically effective amount” means an amount of the compound which is effective to prevent or slow the development of, or to partially or totally alleviate the existing symptoms in a particular condition for which the subject being treated. Determination of a therapeutically effective amount is well within the capability of those skilled in the art. Generally, a therapeutically effective amount can vary with the subject's age, condition, and sex, as well as the severity and type of the medical condition in the subject. Determination of effective amount is within the level of one of ordinary skill in the art.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, GDF-8 binding assays, and immunological assays.

The dosage may be determined by a physician and adjusted, as necessary, to suit observed effects of the treatment. Generally, the compositions are administered so that naringenin is given at a dose from 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg.

In some embodiments, the oral dosage form contains from 70 to 5000 mg/dose naringenin. In some preferred embodiments, the dose is from 100 to 1500 mg naringenin for a 70 kg patient. In some more preferred embodiment, the dose is from 250 to 1100 mg naringenin for a 70 kg patient.

In some embodiments, naringenin is given at a dose from 70 mg/dose to 5000 mg/dose.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. Examples of dosing schedules are administration once a week, twice a week, three times a week, daily, twice daily or three times daily.

To the extent not already indicated, it will be understood by those of ordinary skill in the art that any one of the various embodiments herein described and illustrated may be further modified to incorporate features shown in any of the other embodiments disclosed herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Reagents and Antibodies

Fetal bovine serum (FBS), phosphate-buffered saline (PBS), Dulbecco's Modified Eagle Medium (DMEM), penicillin, streptomycin and trypsin-EDTA were obtained from Invitrogen Life Technologies (Carlsbad, Calif.). Lipoprotein-free FBS was purchased from Biomedical Technologies (Stoughton, Mass.). Insulin was obtained from Eli-Lilly (Indianapolis, Ind.). Oleate, Naringenin, and Brefeldin A were purchased from Sigma-Aldrich Chemicals (St. Louis, Mo.). Immunofluorescence grade paraformaldehyde was purchased from Electron Microscope Sciences (Hatfield, Pa.). OptiMEM basal medium and lipofectamine 2000 were purchased from Invitrogen Life Technologies (Carlsbad, Calif.). SureSilencing shRNA plasmid kit for human ApoB (GFP) was purchased from SuperArray (Frederick, Md.). MTP fluorescent activity kit was purchased from Roar Biomedical (New York, N.Y.). Unless otherwise noted, all other chemicals were purchased from Sigma-Aldrich Chemicals (St. Louis, Mo.). For immunoprecipitation, Protein A-Sepharose was purchased from Invitrogen (Carlsbad, Calif.) while HRP-conjugated goat anti-mouse secondary was purchased from Santa Cruz Biotech (Santa Cruz, Calif.). For immunofluorescence studies, normal donkey serum and secondary F(ab′)2 antibody fragments, ML grade, were obtained from Jackson Immunoresearch (Bar Harbor, Me.). Mouse anti-HCV Core antigen (5 μg/ml) was purchased from US Biological (Swampscott, Mass.). Goat anti-ApoB (10 μg/ml) was purchased from R&D Systems Inc. (Minneapolis, Minn.). Naringenin, β-cyclodextrin (βCD), methyl b-cyclodextrin (mβCD), and 2-hydroxypropyl β-cyclodextrin (HPβCD) were purchased from Sigma-Aldrich Chemicals (St. Louis, Mo.). Caco-2 cells were purchased from the American Type Culture Collection (Rockville, Md.). Unless otherwise noted all chemical were purchased from Invitrogen Life Technologies (Carlsbad, Calif.).

### Example 2

### Cells and Viruses

The Huh-7.5.1 human hepatoma cell line and a plasmid containing the JFH-1 genome were kindly provided by Dr. Chisari (Scripps Research Institute, CA) and Dr. Wakita (National Institute of Infectious Diseases, Tokyo) respectively. Huh-7.5.1 cells were cultured in DMEM medium supplemented with 10% FBS, 200 units/ml penicillin and 200 mg/ml streptomycin in a 5% CO₂-humidified incubator at 37° C. In vitro transcribed genomic JFH-1 RNA was delivered to cells by liposome-mediated transfection as described by Zhong et al. (2005) (8). Infected Huh-7.5.1 cells were passaged every 3 days and used at passage <15. The presence of HCV in these cells and corresponding supernatants were determined by quantitative Polymerase Chain Reaction (qPCR) and immunofluorescence staining. Primary human hepatocytes were purchased from BD Biosciences (San Jose, Calif.) cultured on a collagen-coated 12 well plate in C+H culture media composed of DMEM supplemented with 10% heat-inactivated FBS, 200 U/mL penicillin/streptomycin, 7.5 μg/mL hydrocortisone, 20 ng/mL EGF, 14 ng/mL glucagons and 0.5 U/mL insulin. The media was supplemented with 2% DMSO for long-term culture of the primary cells.

### Example 3

### HCV Secretion

HCV-infected Huh-7.5.1 cells were plated on a 6-well plate at a density of 1×10⁵ cells/cm² and cultured overnight in standard medium. Prior to the beginning of the experiment, the cells were washed 3 times with PBS and cultured with DMEM containing 5% lipoprotein-free FBS. Oleate, Insulin, Naringenin, and Brefeldin A were added at this time as described in the text. Following 24 hours of incubation, the plate was gently agitated to release mechanically bound particles, the media was collected, filtered to remove cellular debris, and stored at −80° C. for further analysis. The attached cells were washed 3 times with PBS, harvested, pelleted and stored at −80° C. for further analysis.

### Example 4

### Co-Immunoprecipitation

The binding of Huh7.5.1-secreted JFH1 particles to ApoB was assessed using co-immunoprecipitation (Co-IP). Anti human ApoB-100 antibody (5 μg) was bound to 100 μl Protein A-Sepharose on ice. 3 ml of JFH1-infected Huh7.5.1 conditioned media (1×10⁶ cells/ml) was added to the mixture which was subsequently rotated for 4 hours at 4° C. The sample was spinned down at 10,000×g in a microcentrifuge and washed 3 times with 50 mM Tris-HCl, pH 7.5, containing 5 mM EDTA. Finally the sample was eluted in 100 μl of 10 mM Tris-HCl, pH 8.5, containing SDS. Protein concentration in the eluted buffer was quantified as described below, 20 μg protein were loaded on a 7.5% Tris-HCL resolving gel. Resolved proteins were transferred to a PVDF membrane and stained against HCV core (0.5 μg/ml).

### Example 5

### HCV Infectivity

The infectivity of the secreted HCV particles was measured as previously described (8). Naïve Huh7.5.1 cells were grown to 80% confluence and exposed to cell culture supernatants diluted 10-fold in culture media. Following 1 hour incubation at 37° C., the media was replaced, and the cells were cultured for 3 additional days. Levels of HCV infection were determined by immunofluorescence staining for HCV core protein. The viral titer is expressed as focus forming units per milliliter of supernatant (ffu/ml).

### Example 6

### Human ApoB ELISA

Huh-7.5.1 and primary human hepatocytes secreted ApoB was detected in the media using ALerCHEK, Inc (Portland, Me.) total human ApoB ELISA kit. Media was diluted 1:10 with the specimen diluent, and the assay was carried out according to manufacturer's directions.

### Example 7

### HCV Core Antigen ELISA

Huh-7.5.1 secreted HCV core antigen was detected in the media using Wako Chemicals (Cambridge, Mass.) ORTHO HCV antigen ELISA kit. Media was used as is, and the assay was carried out according to manufacturer's directions.

### Example 8

### Total Protein Assay

Total protein content of the cells was measured using BioRad Laboratories (Hercules, Calif.) Protein Assay based on the Bardford method. Briefly, cell pellet was lysed in 350 μl Triton X-100 0.1%, and 5 μl samples were loaded on a 96 well plate, and incubated for 15 min with 250 μl Coomassie Blue reagent at room temperature. Absorbance was measured at 595 nm and compared to bovine serum albumin standard.

### Example 9

### Quantitative Real Time RT-PCR

Virus samples collected in each experiment were filtered with a 0.45 μm filter and a volume of 100 μL for each sample was heated at 95° C. for 45 min. The reverse transcription reaction step was performed on a Mastercycler epgrdientS (Eppendorf) instrument using Omniscript and Sensiscript RT Kits (Qiagen). Real time PCR was performed on a Light Cycler LC-24 (Idaho Technology), using SuperScript™ III Platinum® CellsDirect Two-Step qRT-PCR Kits (Invitrogen) for quantitative PCR. For reverse transcription step, 2 μL of sample without RNA extraction were used. For real time PCR, 1 μL of the reverse transcription reactions was used. All reactions were performed according to the manufacturer's instructions using the primers detailed in Table 1.

TABLE 1
PCR Primers

| Gene | Primer | |
|---|---|---|
| HCV | Forward | 5′-GCAGAAAGCGTCTAGCCATGGCGT-3′ |
| 5′ UTR | Reverse | 5′-CTCGCAAGCACCCTATCAGGCAGT-3′ |
| MTP | Forward | 5′-GAGGTTTCTCTATGCCTGTGGATTT-3′ |
| | Reverse | 5′-CCCAGGATTAACTTCTTAGCTTCCA-3′ |
| ACAT1 | Forward | 5′-CAATACAATGGTGGGTGAAGAGAAG-3′ |
| | Reverse | 5′-AAAATCTTTCCTTGTTCTGGAGGTG-3′ |
| HMGR | Forward | 5′-GACCCCTTTGCTTAGATGAAAAAGA-3′ |
| | Reverse | 5′-GGACTGGAAACGGATATAAAGGTTG-3′ |
| Actin | Forward | 5′-GTCGTACCACTGGCATTGTG-3′ |
| | Reverse | 5′-CTCTCAGCTGTGGTGGTGAA-3′ |
| ACAT2 | Forward | 5′-CATGCGGGAGGCTATACAAT-3′ |
| | Reverse | 5′-GTAGATGGTGCGGAAATGCT-3′ |

### Example 10

### Cellular Viability

Viability of both Huh-7.5.1 cells and primary human hepatocytes was studied using Thermo Fisher Scientific (Waltham, Mass.) aspartate aminotransferase (AST) Infinity liquid reagent. Medium samples (15 μ/well) were loaded on a 96 well plate in triplicates, mixed with 150 μl of the AST liquid reagent. Absorbance decay was measured at 340 nm wavelength, with 15 second intervals in a BioRad (Hercules, Calif.) Benchmark Plus spectrophotometer. Values were normalized to the total amount of AST available per culture, which was determined by total cell lysis induced by 1% Triton X-100 for 20 min at room temperature. Cell viability for all conditions reported in the results section was greater than 90%.

### Example 11

### Microsomal Triglyceride Transfer Protein (MTP) Activity Assay

MTP activity was analyzed using an MTP assay kit as previously described (11). The assay is based on a transfer of a fluorescent signal between donor and acceptor particles due to MTP activity. Briefly, confluent Huh7.5.1 cells stimulated with naringenin or carrier control for 24 hours then washed with ice cold PBS and scraped off the dish using a cell scraper. Samples were homogenized by sonication (3×5 sec) in buffer containing protease inhibitors. The MTP assay was performed by incubating 50 μg cellular protein with 10 μl of donor and acceptor solutions in 250 μl total buffer (15 mM Tris pH 7.4; 40 mM NaCl; 1 mM EDTA). Increase in fluoresecent signal was measured over 12 hours at 37° C. at the excitation wavelength of 465 nm and emission wavelength of 538 nm.

### Example 12

### Animal Studies

Male SCID mice (8 weeks old, 20-25 g) were obtained from Charles River Laboratories (Wilmington, Mass.). Animals were treated in accordance with NIH guidelines, and MGH Subcommittee on Research Animal Care. The mice were allowed free access to laboratory chow and water ad labium. Naringenin was dissolved in 0.5% Tween 20 diluted in saline and given by intraperitoneal injection. Two days following the treatment, animals were sacrificed and blood was withdrawn by cardiac puncture. AST and ALT enzyme levels were assessed as described above. Total triglycerides were measured using a kit purchased from Sigma-Aldrich Chemicals (St. Louis, Mo.) according to the manufacturer's instructions.

### Example 13

### Silencing ApoB mRNA

HCV-infected Huh-7.5.1 cells were plated T-25 tissue culture flasks at a density of 1×10⁵ cells/cm² and cultured overnight in standard medium. Prior to silencing, the cells were washed 3 times with PBS and media was replaced with OptiMEM basal medium. SureSilencing shRNA (GFP) plasmids against human ApoB100 as well as shRNA plasmid control (500 ng/ml) were combined with lipofectamine 2000 in OptiMEM and incubated with the cells overnight. SureSilencing shRNA plasmids code for GFP which was used to sort the transfected Huh7.5.1 cells, using FACSAria (BD Biosciences) located at the Partners AIDS Research Center. Transfected cells (10% of the total population) were sorted directly into a 12-well plate and allowed to adhere overnight. Culture media was conditioned by the transfected cells for 24 hours and analyzed as described above.

### Example 14

### Immunofluorescence Microscopy

Huh-7.5.1 cells were washed 3 times with PBS and fixed in 4% EM-grade paraformaldehyde for 10 minutes at room temperature. Slides were then washed with PBS and incubated in 100 mmol/L glycine for 15 minutes to saturate reactive groups. Samples were permeabilized for 15 minutes with 0.1% Triton X-100, blocked for 30 minutes with 1% bovine serum albumin and 5% donkey serum at room temperature, and stained with primary antibodies overnight at 4° C. After additional washes with PBS, samples were stained with fluorescently tagged secondary antibodies for 45 minutes at room temperature.

### Example 15

### LC-MS Detection of Naringenin

LC-MS analysis was performed on an Agilent Technologies series 1100 LC-MSD system (Santa Clara, Calif.), which included an Agilent 1100 quaternary pump, autosampler, column oven, on-line vacuum degassor, and single quadrupole mass spectrometer equipped with electrospray ion source (ESI).

Mass spectrometry conditions: Electrospray ionization (ESI), positive, selected ion monitoring scan (SIM); SIM: naringenin m/z 273.1; IS (hesperetin) m/z 303.1. LC conditions: Eclipse XDB-C18 column (4.6×150 mm, 5.0 μm). The mobile phase was composed of methanol-water with 0.1% formic acid (65:35, v/v). The isocratic flow rate was set at 0.8 ml/min and injection volume was only 10 μl.

To each 100 μl of rat serum sample, 100 μl of 0.1N sodium acetate (pH=5.0) and 100 μl of β-glucuronidase enzyme (5000 units/mL, type HP-2 from Helix Pomatia) were added and votexed for 5 seconds. This process hydrolyzes the conjugated form of naringenin to determine total naringenin in plasma. After addition of 20 μl IS buffer solution (5 μg/mL), the sample was then incubated at 37° C. water bath for 18 h.

The sample was extracted with 0.8 mL of ethyl acetate after 18 h incubation, and centrifuged at 13000 rpm for 10 min. The supernatant was collected and evaporated to dryness under nitrogen at room temperature. The residue was reconstituted with 100 μl of mobile phase and filtered through a micro nylon n filter (0.45 μm). 10 μl of the filtrate was forwarded to LC-MS analysis. A calibration curve was established and QC samples conducted (data not shown). Data acquisition was performed using ChemStation software (Agilent). Linear regression (weighted by 1/x) between serum concentration and peak area ratio of naringenin to IS was constructed using SPSS11.0 statistical software. The concentrations of naringenin in samples were calculated by interpolation of the linear equation.

### Example 16

### Liver Histology

Formalin-fixed, paraffin-embedded liver, intestine, and kidney samples were sectioned at 4 μm and stained with hematoxylin & eosin (H&E). Histological characterization was performed by a blinded observer using standard assessment of damage.

### Example 17

### Statistics

Data are expressed as the mean±standard deviation. Statistical significance was determined by a one-tailed Student's t-test. A P-value of 0.05 was used for statistical significance.

### Example 18

### Huh7.5.1-Secreted HCV is Bound to ApoB

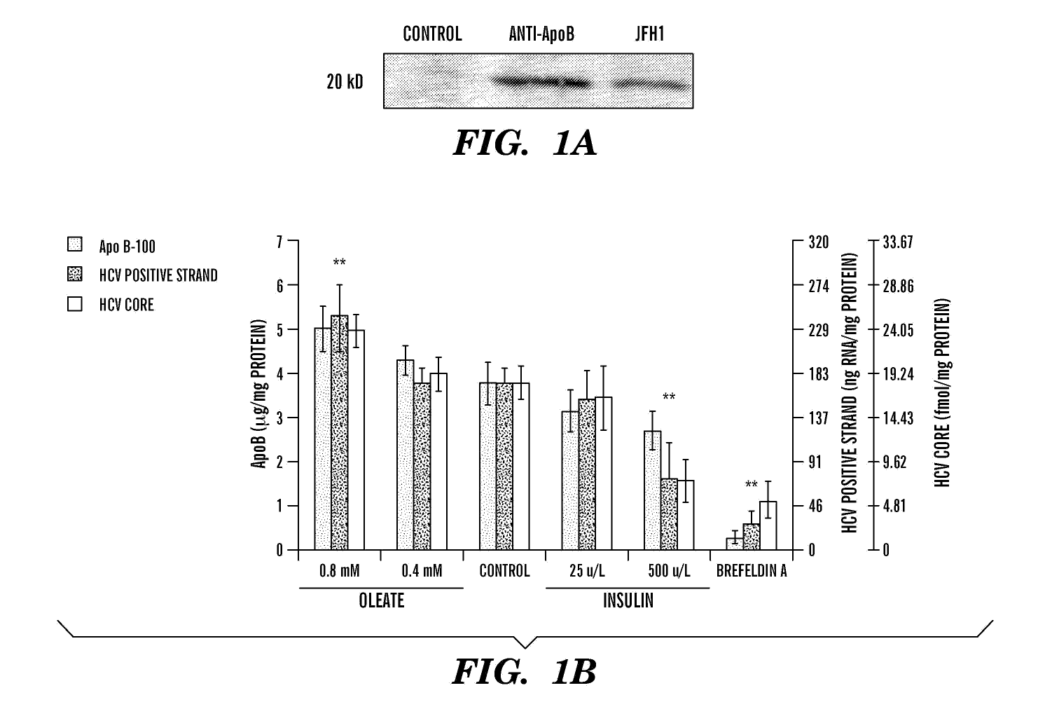

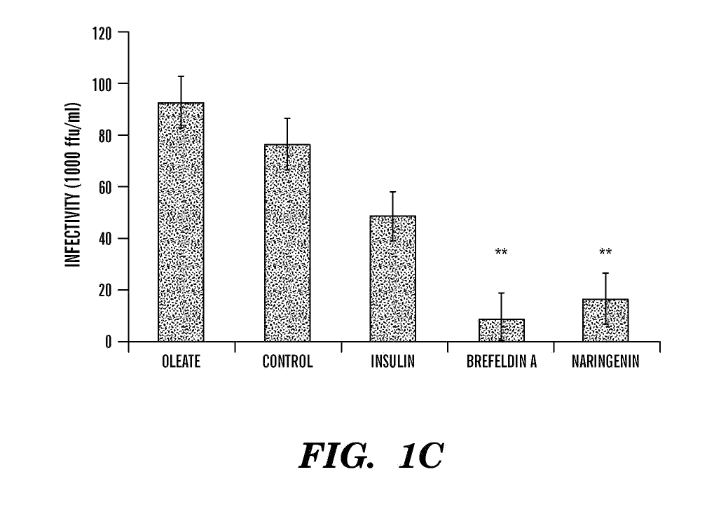

Recent evidence suggests that HCV binds to low density particles prior to virus egress (9) and that viral secretion requires both ApoB expression and vLDL assembly to occur (10). Therefore, HCV secreted by the JFH1/Huh7.5.1 full viral lifecycle model could potentially be secreted while bound to vLDL. To determine if Huh7.5.1-produced HCV is bound to vLDL, we immunoprecipitated Huh7.5.1 conditioned media against human ApoB antibodies and detected bound HCV core protein in the eluted sample. The results presented in FIG. 1A demonstrate that HCV core protein is bound to ApoB-100 in our samples. HCV core could not be detected when the sample was precipitated against irrelevant antibody (control), but easily detected in the cell media (JFH1).

### Example 19

### HCV Secretion Mirrors that of vLDL

The interaction between HCV and ApoB suggests that the virus might be actively secreted by the cells while bound to vLDL. However, the interaction between these particles might also occur outside the cell. To determine if HCV is being actively secreted by the cells while bound to vLDL, we studied viral secretion in response to oleate and insulin stimulation which were previously shown to oppositely modulate ApoB secretion in culture(12). FIG. 1B shows ApoB, HCV core, and HCV positive strand RNA secretion by Huh-7.5.1 cells infected with the JFH-1 virus. As expected, ApoB secretion is significantly upregulated by oleate (P=0.0023 N=5) and downregulated by insulin (P=0.0073 N=5) in a dose-dependent manner. Similarly, HCV core protein secretion is significantly upregulated by oleate (P=0.0073 N=3) and downregulated by insulin (P=0.0223 N=3) in a dose dependent manner. The secretion of HCV positive strand RNA, measured by qPCR, follows the same path. However, intracellular levels of HCV RNA remained unchanged following both treatments.

Brefeldin A is a commonly used toxin which disrupts communication between the endoplasmic reticulum and the Golgi, inhibiting the active secretion of proteins (12, 13). Not surprisingly, the addition of brefeldin A (2.5 μg/ml) blocked ApoB secretion (P=0.0001 N=5). Interestingly, brefeldin A significantly inhibits the secretion of HCV core protein (P=0.0021 N=4), and HCV positive strand RNA (P=0.0006 N=3). To assess whether the changes in HCV core protein and RNA secretion correlate with changes of viral infectivity in the cell supernatant, we measured the ability of the secreted virus to infect naïve Huh7.5.1 cells. FIG. 1C shows that the infectivity of the cell supernatant increased following oleate stimulation, decreased due to insulin, and was strongly inhibited following brefeldin A stimulation by 89%±10% (P=0.001 N=3). These results suggest that HCV is being actively secreted by the cells, perhaps while bound to vLDL.

### Example 20

### HCV Core Antigen Colocalizes with ApoB

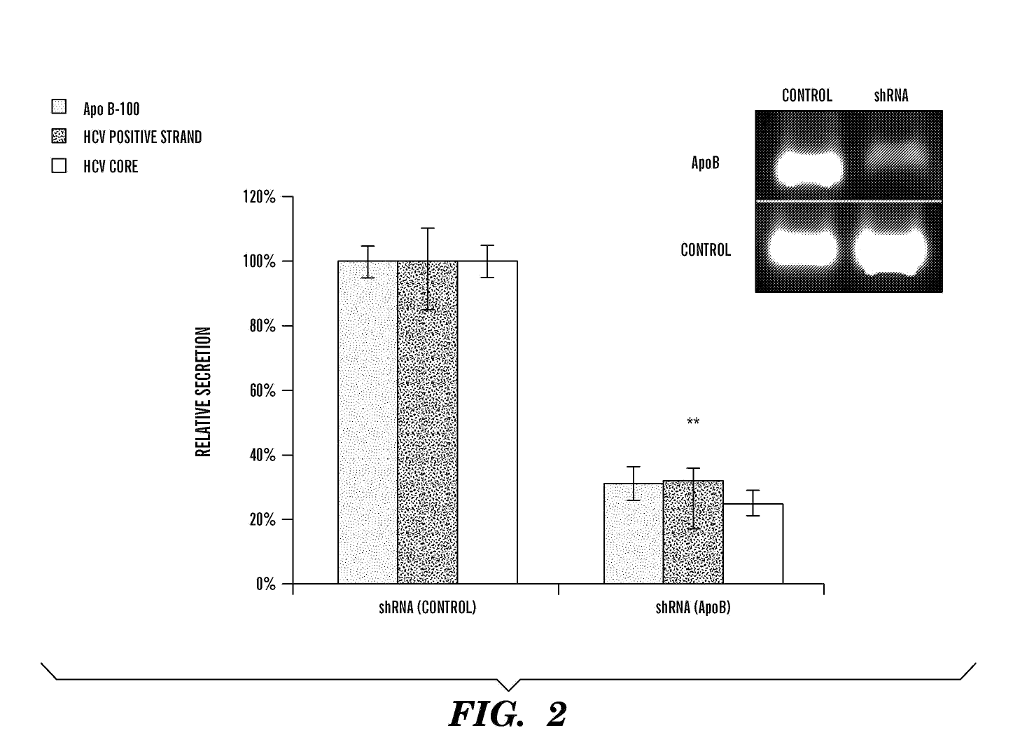

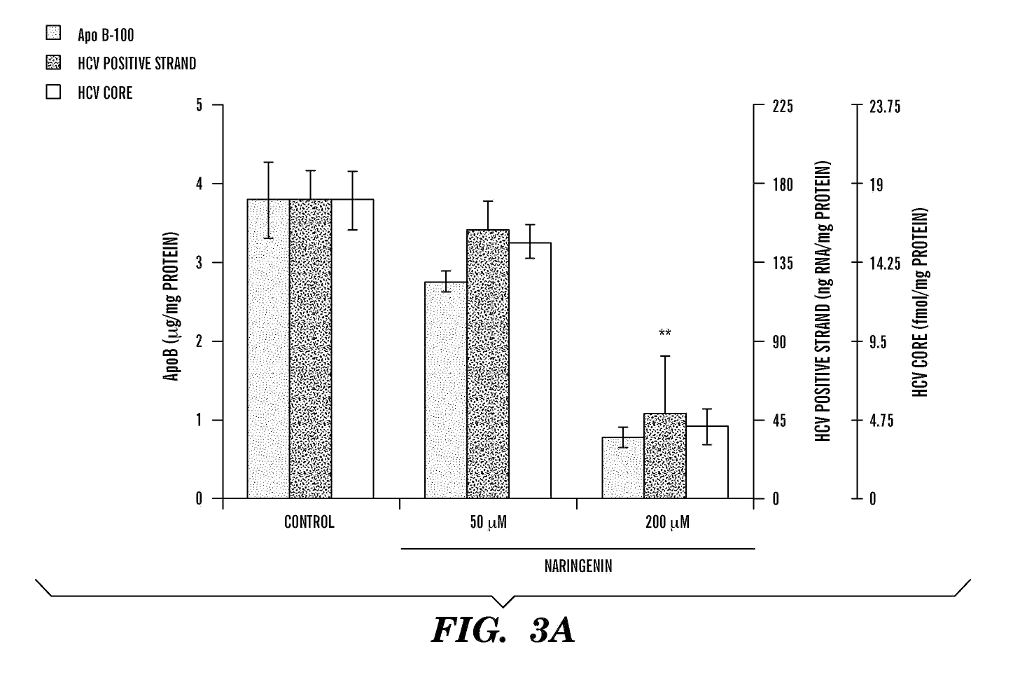

Previously, HCV's core protein was shown to associate with apoAII (4) and lipid droplets in HepG2 cells(5) cells over-expressing the core protein. Just recently, Huang et al. demonstrated that HCV Core protein colocalizes with ApoB in a chromosomally integrated cDNA model of HCV (10). To ascertain if HCV's core protein associates with ApoB in JFH-1 virus infected Huh-7.5.1 cells, we double-stained Huh7.5.1 cells two days post infection by immunofluorescence for both viral and native proteins. FIG. 2 demonstrates the colocalization of HCV's core and ApoB100 in infected cells. HCV's core protein associates with areas in the cytoplasm which are positive to ApoB100. However, we note that although the proteins appear to be closely associated, we fail to find a one to one correspondence between the viral and native proteins in our model of the full viral lifecycle.

The association between ApoB100 and HCV core protein as well as previous data suggests that HCV might be ‘tagging along’ ApoB secretion. Therefore, silencing ApoB production in the cell might decrease HCV secretion. FIG. 2D demonstrates a 69±6% decrease in ApoB secretion following transfection with SureSilencing shRNA (P=0.0001 N=3). Interestingly, HCV core protein secretion was significantly decreased by 75±4% at the same time (P=0.0002 N=3). HCV positive strand RNA secretion was also significantly decreased by 69±4% (P=0.0015 N=3).

### Example 21

### HCV Secretion is Inhibited by Naringenin

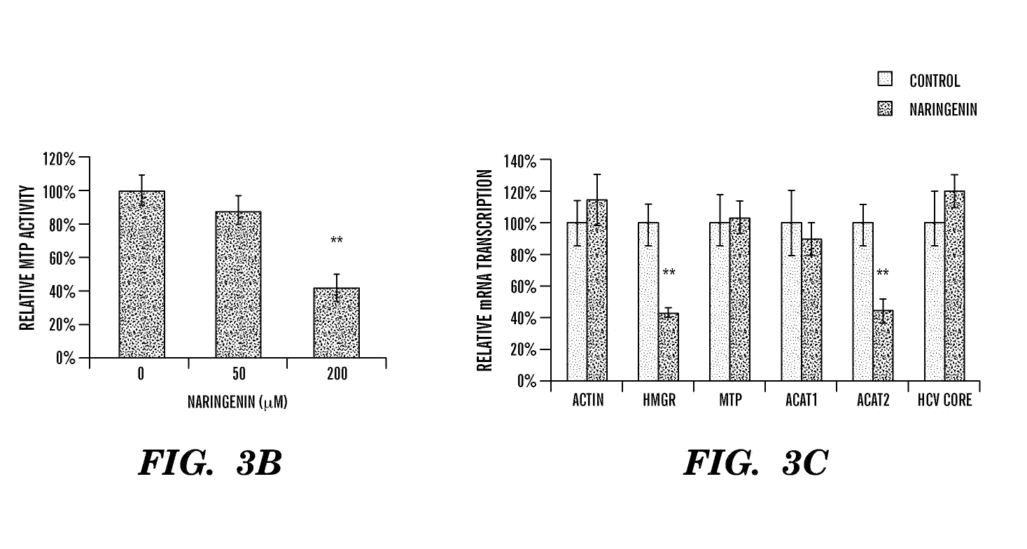

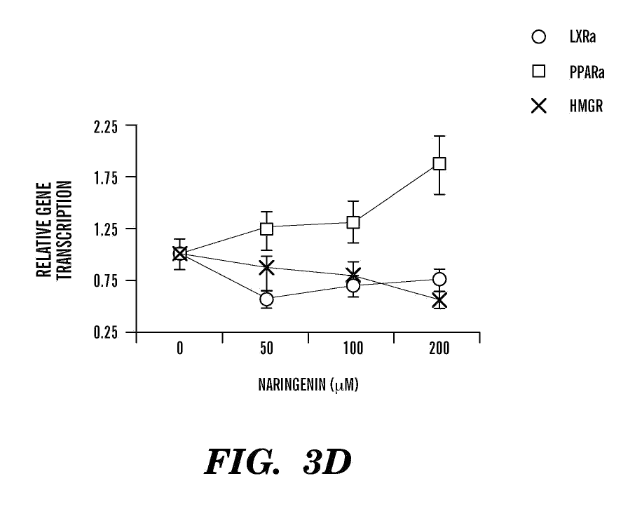

Naringenin is a grapefruit flavonoid previously shown to reduce cholesterol levels both in vivo(14) and in vitro(15). It is thought that naringenin inhibits ApoB secretion by reducing the activity and expression of microsomal triglyceride transfer protein (MTP) and acyl CoA:cholesterol acyltransferase (ACAT) (15, 16). To assess if naringenin inhibits HCV secretion in a similar manner we cultured infected Huh-7.5.1 cells in the presence of naringenin for 24 hours. FIG. 3A demonstrates that naringenin inhibits the secretion of HCV core (P=0.0001 N=6) and HCV positive strand RNA (P=0.0006 N=5) in a dose dependent manner. At 200 μM concentration, naringenin inhibited HCV secretion by 80%±10%. Interestingly, intracellular levels of HCV positive strand RNA (FIG. 3C) as well as intracellular HCV core protein expression (Supplemental FIG. S1) remained unchanged. To assess whether the naringenin-induced inhibition in HCV core protein and RNA secretion correlate with changes of viral infectivity in the cell supernatant, we measured the ability of the secreted virus to infect naïve Huh7.5.1 cells. FIG. 1C shows that the infectivity of the cell supernatant was strongly inhibited following naringenin stimulation by 79%±10% (P=0.0018 N=3).

Although the activity of naringenin has been described in uninfected cells (15, 17, 18), it has yet to be characterized in HCV infected cells. FIG. 3B demonstrates that naringenin inhibits MTP activity in a dose dependent manner. At 200 μM concentration, MTP activity was reduced by 58%±8% (P=0.0012 N=3). In addition, we demonstrate that naringenin induces significant changes in hepatic gene transcription measured by qPCR (FIG. 3C). HMGR transcription was reduced by 57%±3% (P=0.010 N=3), while ACAT2 was reduced by 55%±7% (P=0.016 N=3). In contrast, the mRNA levels of actin, MTP, ACAT1, as well as that of HCV remained unchanged.

The myriad effects of naringenin on cellular metabolism suggest it affects underlying transcriptional regulatory elements. PPARα and LXRα are ligand-activated transcription factors which control much of the fasted-to-fed transition and were previously shown to be important in the development of inflammation and insulin resistance. Here we demonstrate that naringenin dose-dependently induces PPARα transcription in infected cells, relative to control (FIG. 3D). Following a short term 24 hours stimulation of Huh7.5.1 cells PPARα transcription increased by 86%, while LXRα was decreased by 25%. PPARα is the target of fibrates and plays a major role in the downregulation of inflammation, increasing sensitivity to insulin, and modulating dyslipidemia.

### Example 22

### Naringenin does not Display Hepatic or In Vivo Toxicity

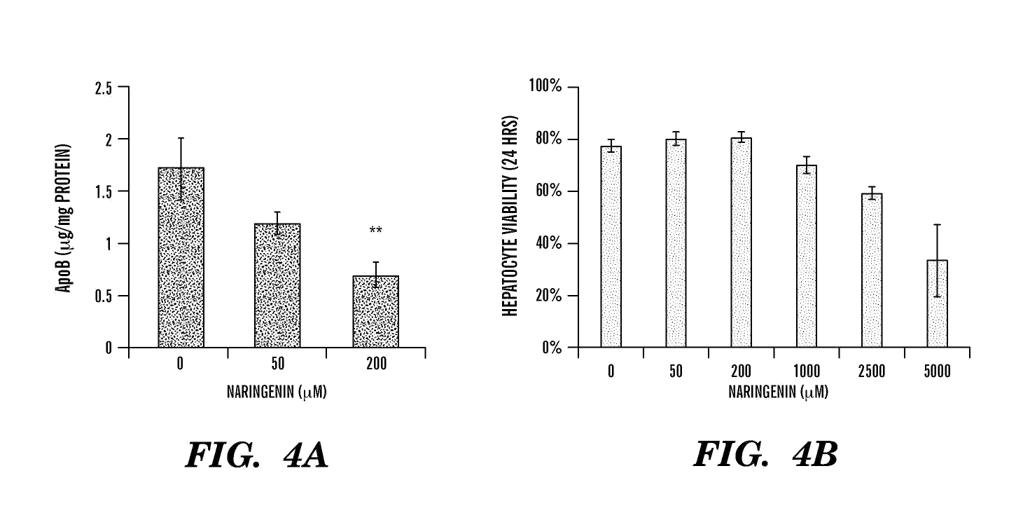

To assess the potential of naringenin-based treatment, we measured ApoB secretion in primary human hepatocytes following 24 hours stimulation with naringenin. FIG. 4A demonstrates a dose-dependent decrease in ApoB secretion following naringenin stimulation. At 200 μM naringenin, ApoB secretion was reduced by 60%±7% (P=0.007 N=3). The viability of primary human hepatocytes exposed to increasing concentrations of naringenin is shown in FIG. 4B. Human hepatocyte viability was 81%±3% at 200 μM naringenin and was not judged to be statistically different than control (78%±3%). Human hepatocyte viability dropped significantly only at naringenin concentrations greater than 1000 μM.

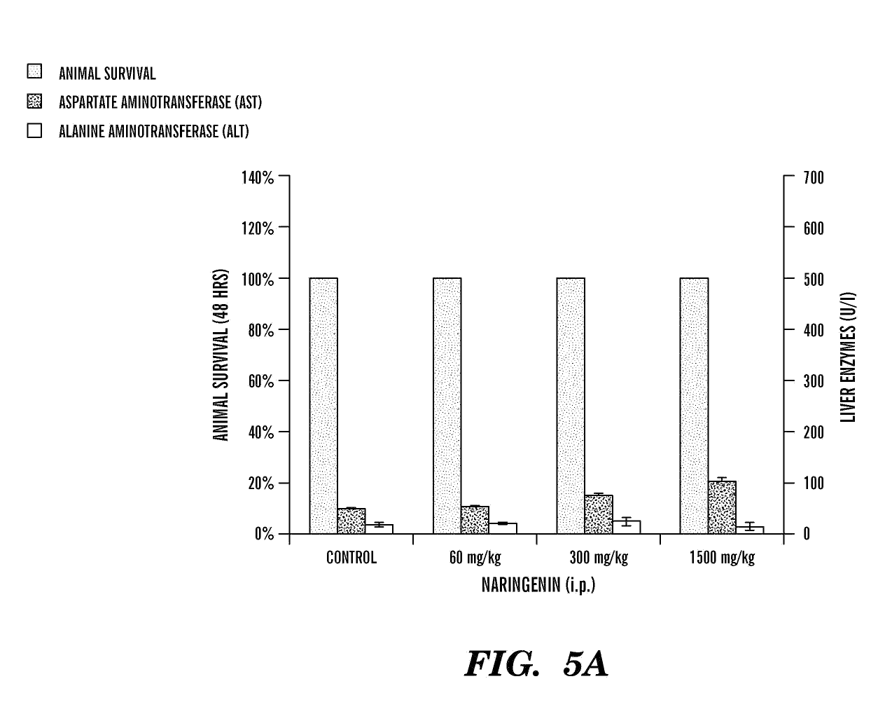

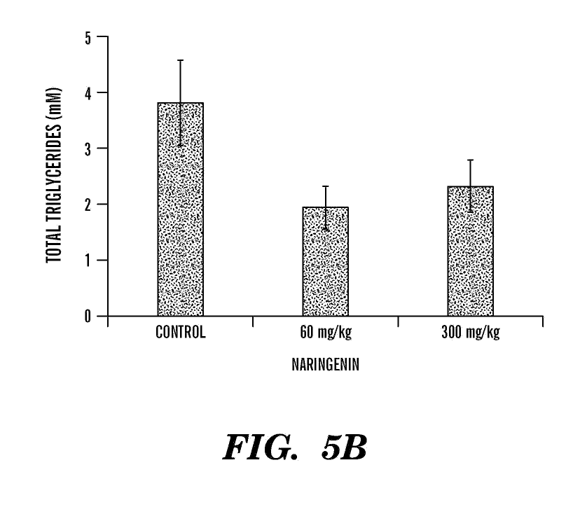

To further assess naringenin potential, we delivered naringenin by intraperitoneal injection to 8 week old male SCID mice at concentrations of 60, 300, and 1500 mg/kg (approximately 200, 1000, 5000 μM). Animal survival was not affected by naringenin at these doses. To discern if liver damage occurred we measured levels of aspartate aminotransferase (AST) and alanine aminotransferase (ALT) in the animal's plasma 48 hrs following injection. FIG. 5 demonstrates that there was no elevation of ALT levels in all conditions. AST levels appeared to increase, but remained under 100 U/l even at the highest dose. To assess naringenin ability to reduce circulating vLDL levels we measured total triglycerides levels in animal plasma. FIG. 5a demonstrates a decrease in triglycerides following naringenin injection.

### Example 23

### Long-Term Treatment of HCV Infected Cells with Naringenin Compared with Interferon α (IFNα)

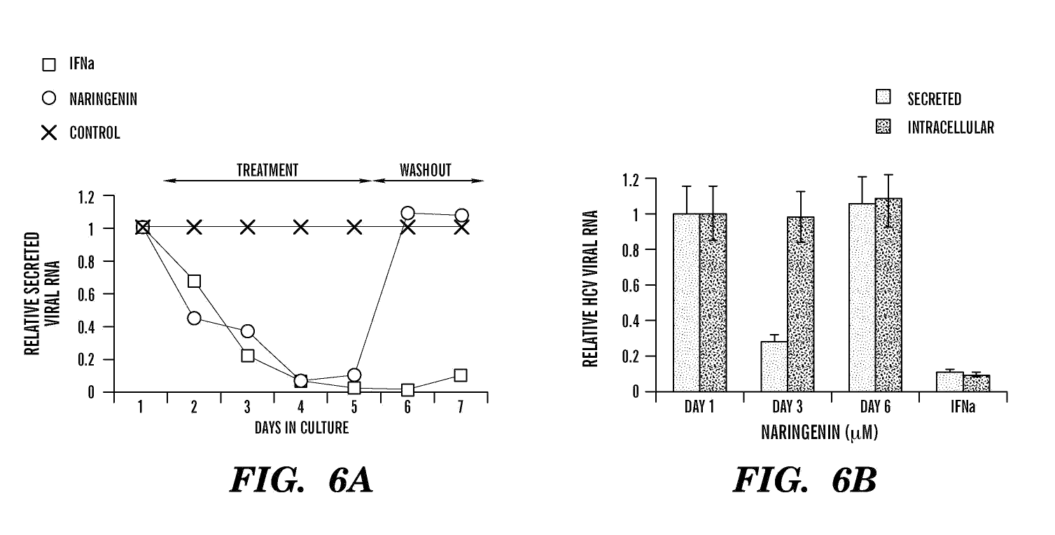

Our previously published results demonstrated that short-term stimulation with naringenin resulted in 80% inhibition of HCV secretion/infectivity³⁶. However, this experiment was carried out on cells continuously producing the virus. Long-term treatment could potentially have a greater effect. FIG. 6A shows HCV RNA secretion by infected Huh7.5.1 cells during a four-day treatment with 200 μM naringenin. This treatment is compared to the current standard-of-care, 1000 i.u. of Interferon α (IFNα). Both treatments lowered HCV RNA to a similar level, 96±5% and 93±5% respectively. However, at this dose naringenin didn't appear to effect HCV replication (FIG. 6B), and during the washout period, HCV RNA secretion was not different than untreated control. IFNα treatment predictably inhibited both viral replication and secretion at day 6 of culture. As IFNα treatment is associated with severe side-effects and the potential acquisition of viral resistance, this data supports the concept of naringenin-based therapy.

### Example 24

### Increased Solubility of Naringenin/Cyclodextrin Complexes

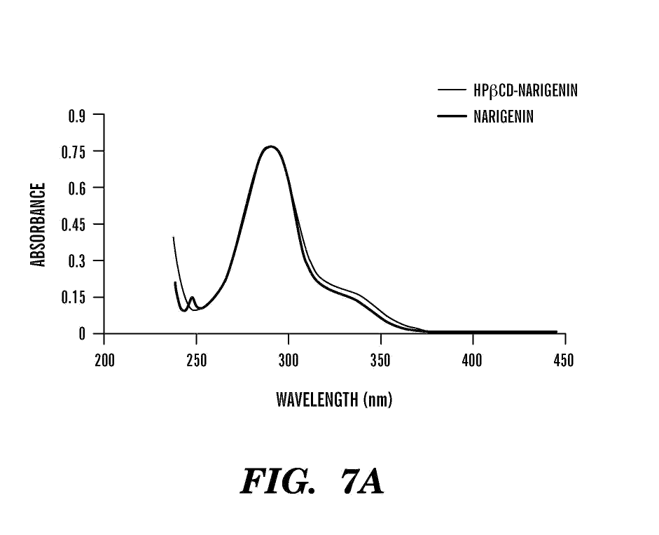

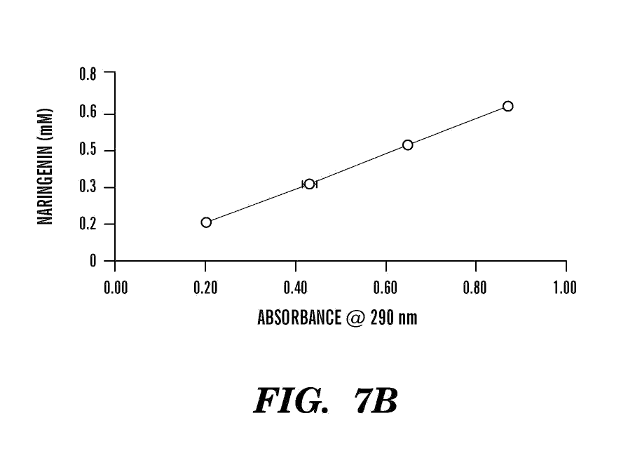

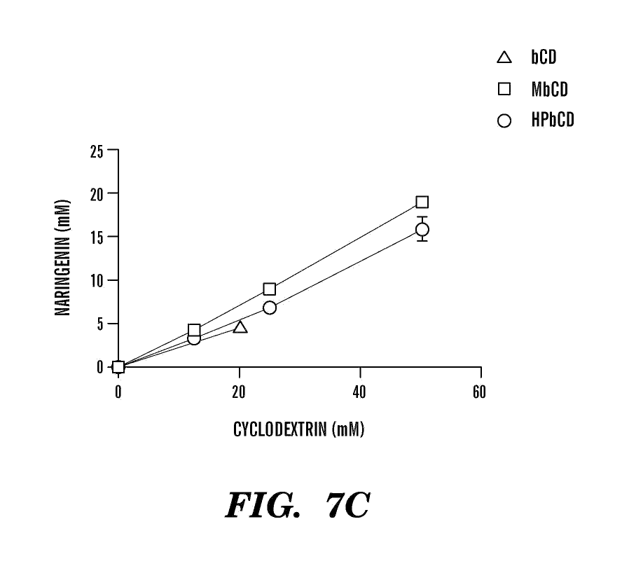

One of the challenges of developing an oral delivery method for naringenin is its low intestinal absorption (about 6%)³⁷. Recently, the flavonoid-glycoside rutin was shown to have enhanced absorption in beagle dogs following complexation with β-cyclodextrin³⁸, a cyclic oligosaccharide which forms host-guest complexes with hydrophobic molecules such as steroids, increasing their solubility. Stock solutions of βCD, mβCD, and HPβCD were prepared in distilled water. None of the cyclodextrins absorbed at 290 nm for concentrations from 0-50 mM (data not shown). Next, excess amounts of naringenin powder were added to solutions containing variable amounts of each cyclodextrin, vortexed, and incubated with shaking at 37° C. for 3-5 days. Naringenin-cyclodextrin solutions were filtered through a 0.45 μm membrane to remove the undissolved naringenin, diluted by 20 or 50-fold, and its absorbance measured at 290 nm using Nanodrop ND1000. Dissolved naringenin concentrations were determined using the UV absorbance at 290 nm and the calibration curve. Cyclodextrin, doesn't absorb at this range nor change naringenin's UV spectra (data not shown). Naringenin/Cyclodextrin solubility curve is shown in FIG. 7. We note that MβCD is used to disrupt lipid rafts, and therefore might have deleterious effects on cells, while the solubility of βCD is limited to about 20 mM. However, HPβCD increased naringenin's solubility from 0.04 mM to 15 mM.

As expected, naringenin solubility in water was 36 μM±1 μM, consistent with values reported in literature. Upon addition of cyclodextrins, the amount of solubilized naringenin increased considerably, as summarized in Table 2. The three βCDs solubilized naringenin in decreasing order mβCD>HPβCD>βCD.

TABLE 2
Naringenin solubility in cyclodextrin solutions.

| | Max. Naringenin | Corresponding | Fold increase | |
|---|---|---|---|---|
| | Conc. (mM) | CD Conc. (mM) | in solubility | K |
| βCD | 4.8 ± 0.3 | 20 ± 1 | 132 | |
| mβCD | 19 ± 0.9 | 50 ± 2.5 | 526 | |
| HPβCD | 15.8 ± 1.4 | 50 ± 2.5 | 437 | |

### Example 25

### Naringenin/Cyclodextrin Complex Demonstrates Dramatically Enhanced Intestinal Transport

The Caco-2 cell line is an immortalized line of human epithelial colorectal adenocarcinoma cells, which is widely used by the pharmaceutical industry to predict the absorption rate of candidate drug compounds across the intestinal epithelial cell barrier. Caco-2 cells were grown and allowed to form confluent monolayers on transwell filters for 21 days as previously described³⁹. In detail, Caco-2, human epithelial colorectal adenocarcinoma cells were cultured in tissue culture flasks (Becton Dickinson and Co., Lincoln Park, N.J.). The growth medium was Dulbecco's modified Eagle's medium supplemented with 10% fetal bovine serum, 1% nonessential amino acids, and 4 mM glutamine without antibiotics. The monolayer cultures were grown in a CO₂ incubator (5% CO₂) at 37° C. The cells were harvested with 0.25% trypsin and 0.2% EDTA (0.5 to 1 min at 37° C.), resuspended, and seeded into a new flask. Cells between 30 to 53 passages were used.

For the transport studies, Caco-2 cells were seeded on Transwell (0.4-μm pore size, 1-cm² growth area; Corning Costar Co.) at a cell density of 1×10⁵ cells/filter. Cell growth and maintenance was kept as previously described (98). The cell monolayer was fed fresh growth medium every 2 days and was then used on Day 21 for the transport experiments. To evaluate the integrity of the monolayer, transepithelial HBSS supplement with 20 mM d-glucose and 10 mM HEPES (pH 7.35) was used as the transport medium. To determine the amount of drug crossing the polarized Caco-2 cell monolayer from the donor to the receiver (i.e., apical to basolateral), the Caco-2 cells were rinsed twice with pre-warmed transport medium and were incubated by pre-warmed transport medium 0.2 ml for apical chamber and 0.5 ml for basolateral chamber at 37° C. for 30 min. A 60 mg/ml (1% DMSO in HBSS) stock solution of test compounds, either naringenin or HPβCD-naringenin, was added and samples from both apical and basolateral were taken (30 μl) at different time points: 30, 60, 120, 150 180, 240, and 300 min. The integrity of the culture was confirmed by microscopy and by detecting fluorescently labeled cells using Lucifer Yellow (60 μM) as a standard. The concentrations of Naringenin or HPbCD-Neringenin were determined as described and plotted as a concentration on the basolateral side vs. time. Concentrations were corrected by the dilution factor as fresh buffer was added after sampling.

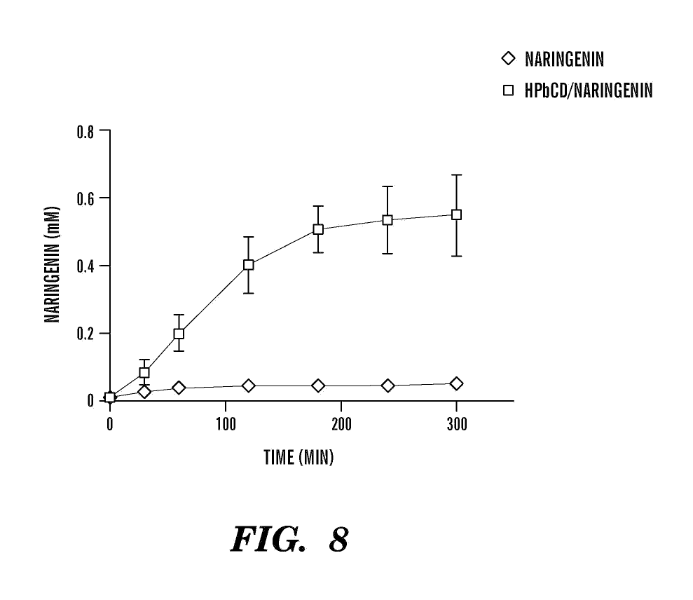

In one experiment, 11 nM of naringenin, either alone or in complex form with 45 mM HPβCD, was added to the top chamber. Sample were taken from both top apical chamber and bottom basal chamber at different time intervals and assayed for concentrations of naringenin. FIG. 8 demonstrates the basal accumulation of naringenin over time. As can be seen the transport of naringenin across the intestinal barrier is extremely limited, reaching a maximal of 0.05 mM concentration after 2 hours. On the other hand, the naringenin/HPβCD complex reaches a concentration of 0.50 mM after 3 hours of incubation.

In the presence of HPβCD, the concentration of naringenin increased from 0.04 μM±0.02 μM to 0.51 μM±0.07 μM, representing an 11-fold enhancement of transport across the Caco-2 monolayer. This 11 fold increase in intestinal absorption makes oral delivery of naringenin possible. These results are especially encouraging as the effective dose of naringenin in cell culture is 0.20 mM, which is beyond its solubility in water (<0.05 mM) but within the range of is cyclodextrin complex to solubilize and transport across the intestinal barrier. To the best of our knowledge this is the first demonstration of enhanced flavonoid transport across a human intestinal barrier using cyclodextrin. The integrity of the Caco-2 monolayer was verified at the end of the experiment by measuring the transport of Lucifer yellow, and found to be similar for both control and treatment (data not shown).

### Example 26

### Preparation of Naringenin/Cyclodextrin Complex

Stock solutions of naringenin were prepared in ethanol. A calibration curve was prepared by measuring the UV absorbance of the naringenin stock solutions (0.1-0.6 mM) at 290 nm using a ND-1000 spectrophotometer (NanoDrop Technologies, Rockland, Del., USA). Standard deviations between triplicate measurements were less than 5%.

Complexes are formed by dissolving β-cyclodextrin in PBS (phosphate buffer saline) and adding measured amount of naringenin. The mixture is then incubated at 37° C. under constant shaking for 24 to 48 hours to allow the complex to form.

For the specific case of the solubility curve, the solution was filtered through a 0.22 uM pore syringe filter to remove undissolved naringenin and final concentration was determined by UV absorbance at 290 nm.

### Example 27

### Oral Administration to Animals

Adult male Sprague-Dawley rats were purchased from Charles Rivers Laboratories (Wilmington, Mass.). Upon arrival each rat was isolated for 5 days towards adaptation to the new environment. Animals were housed under 12 h cycle of day/night with free access to drinking water and fed ad libitum. Briefly, rats weighing between 280 and 300 g were anaesthetized using intraperitoneal injections of ketamine and xylazine at 110 and 0.4 mg/kg, respectively. Shortly, the left carotid artery was cannulated using a 0.76-mm diameter×60-cm length heparanized catherter. The catheter was tunneled subcutaneously from the opening made in the anterior face of the neck to the dorsal site of the neck and permanently anchored in the skin. The catheter was secure by the use of a rat jacket. Animals were placed in their cages during the term of the study. Animals were orally administered with either 20 mg/kg body weight of naringenin in either water or complexed with 320 mg/kg body weight HPβCD with using a rat oral gavage (18 G×1½″ plastic feeding tube from Instech Laboratories, Inc, PA, USA). Blood samples (0.5 ml) were collected at 0, 15, 30, 60, 120, 240, 360, 510, and 600 min from the carotid artery using the previously placed catheter. In two additional experiments, animals were placed in metabolic cages and urine was collected a pooled for the duration of the experiment.

Immediately after collection plasma was separated and stored at −80° c. for further analysis. At the conclusion of the experiment, all animals were sacrificed, and liver, kidney, and bowel specimens were collected for histology. In an additional experiments, animals were placed in metabolic cages and urine was collected a pooled for the duration of the experiment. Total naringenin (flavonoid and glycoside) was determined by LC-MS as described above.

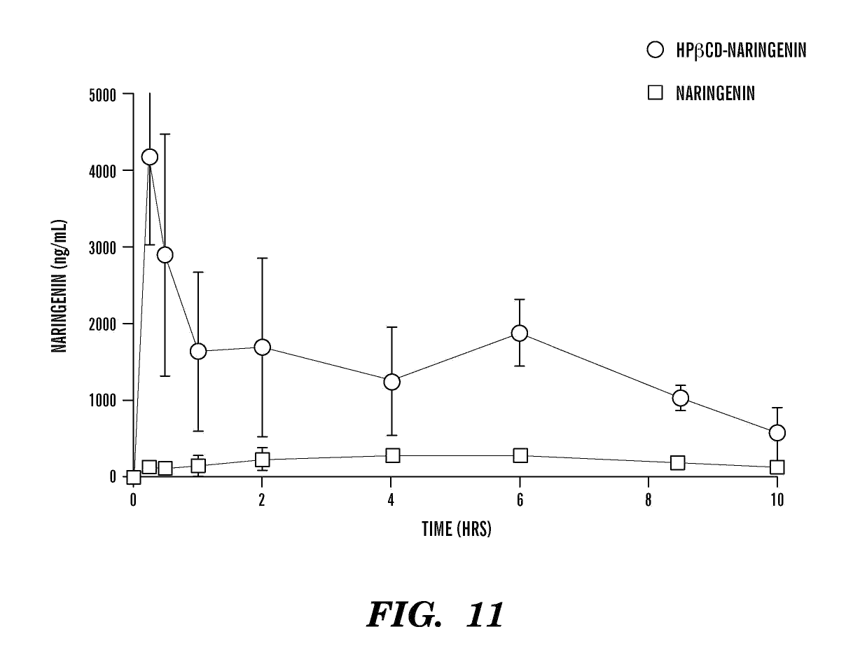

Addition of naringenin significantly affected the plasma concentration versus time profile of naringenin (FIG. 11). Complexation with HPβCD significantly increased the AUC₀₋₁₀ of naringenin from 2.0±0.5 hr*μg/mL to 15.0±4.9 hr*μg/mL representing a 7.4-fold increase in bioavailability (p=0.005 n=3). Naringenin maximal concentration, Cₘₐₓ increased from 4.3±1.2 μg/mL to 0.3±0.1 μg/mL representing a 14.6-fold increase (p=0.002 n=3). Finally, analysis of urine samples in two animals demonstrated renal clearance of 4.2±1%.

Histological examination of liver, kidneys, and the intestine showed no gross pathological changes or significant histological changes (data not shown).

### REFERENCES

1. Guidotti L G, Chisari F V. Immunobiology and Pathogenesis of Viral Hepatitis. The Annual Review of Pathology: Mechanisms of Disease 2006; 1:23-61.
2. Thomssen R, Bonk S, Propfe C, Heermann K H, Kochel H G, Uy A. Association of hepatitis C virus in human sera with beta-lipoprotein. Med Microbiol Immunol. 1992; 181:293-300.
3. Monazahian M, Kippenberger S, Muller A, Seitz H, Bohme I, Grethe S, Thomssen R. Binding of human lipoproteins (low, very low, high density lipoproteins) to recombinant envelope proteins of hepatitis C virus. Med Microbiol Immunol 2000; 188:177-184.
4. Sabile A, Perlemuter G, Bono F, Kohara K, Demaugre F, Kohara M, Matsuura Y, et al. Hepatitis C virus core protein binds to apolipoprotein AII and its secretion is modulated by fibrates. Hepatology 1999; 30:1064-1076.
5. Barba G, Harper F, Harada T, Kohara M, Goulinet S, Matsuura Y, Eder G, et al. Hepatitis C virus core protein shows a cytoplasmic localization and associates to cellular lipid storage droplets. PNAS 1997; 94:1200-1205.
6. Kapadia S B, Chisari F V. Hepatitis C virus RNA replication is regulated by host geranylgeranylation and fatty acids. PNAS 2005; 102:2561-2566.
7. Wakita T, Pietschmann T, Kato T, Date T, Miyamoto M, Zhao Z, Murthy K, et al. Production of infectious hepatitis C virus in tissue culture from a cloned viral genome. Nature Medicine 2005; 11:791-796.
8. Zhong J, Gastaminza P, Cheng G, Kapadia S, Kato T, Burton D R, Wieland S F, et al. Robust hepatitis C virus infection in vitro. PNAS 2005; 102:9294-9299.
9. Gastaminza P, Kapadia S B, Chisari F V. Differential Biophysical Properties of Infectious Intracellular and Secreted Hepatitis C Virus Particles. Journal of Virology 2006; 80:11074-11081.
10. Huang H, Sun F, Owen D M, Li W, Chen Y, M M J G, Ye J. Hepatitis C virus production by human hepatocytes dependent on assembly and secretion of very low-density lipoproteins. PNAS 2007; 104:5848-5853.
11. Perlemuter G, Sabile A, Letteron P, Vona G, Topilco A, Chretien Y, Koike K, et al. Hepatitis C virus core protein inhibits microsomal triglyceride transfer protein activity and very low density lipoprotein secretion: a model of viral-related steatosis. FASEB J. 2002; 16:185-194.
12. Dixon J L, Ginsberg H N. Regulation of hepatic secretion of apolipoprotein B-containing lipoproteins: information obtained from cultured liver cells. Journal of Lipid Research 1993; 34:167-179.
13. Misumi Y, Misumi Y, Miki K, Takatsuki A, Tamura G, Ikehara Y. Novel blockade by brefeldin A of intracellular transport of secretory proteins in cultured rat hepatocytes. Journal of Biological Chemistry 1986; 261:11398-11403.
14. Kurowska E, Borradaile N, Spence J D, Carroll K K. Hypocholesterolemic effects of dietary citrus juices in rabbits. Nutr. Res. 2000; 20:121-129.
15. Allister E M, Borradaile N M, Edwards J Y, Huff M W. Inhibition of microsomal triglyceride transfer protein expression and apolipoprotein B100 secretion by the citrus flavonoid naringenin and by insulin involves activation of the mitogen-activated protein kinase pathway in hepatocytes. Diabetes 2005; 54:1676-1683.
16. Wilcox L J, Borradaile N M, Dreu LEd, Huff M W. Secretion of hepatocyte apoB is inhibited by the flavonoids, naringenin and hesperetin, via reduced activity and expression of ACAT2 and MTP. Journal of Lipid Research 2001; 42:725-734.
17. Borradaile N M, Dreu LEd, Barrett P H R, Huff M W. Inhibition of hepatocyte apoB secretion by naringenin: enhanced rapid intracellular degradation independent of reduced microsomal cholesteryl esters. Journal of Lipid Research 2002; 43.
18. Borradaile N M, Dreu LEd, Barrett P H R, Behrsin C D, Huff M W. Hepatocyte ApoB-Containing Lipoprotein Secretion Is Decreased by the Grapefruit Flavonoid, Naringenin, via Inhibition of MTP-Mediated Microsomal Triglyceride Accumulation. Biochemistry 2003; 42:1283-1291.
19. Guidotti L G, Chisari F V. Immunobiology and Pathogenesis of Viral Hepatitis. Annual Review of Pathology: Mechanisms of Disease 2006; 1:23-61.
20. Andre P, Perlemuter G, Budkowska A, Bre'chot C, Lotteau V. Hepatitis C Virus Particles and Lipoprotein Metabolism. Semin Liver Dis. 2005; 25:93-104.
21. Lohmann V, Korner F, Koch J, Herian U, Theilmann L, Bartenschlager R. Replication of subgenomic hepatitis C virus RNAs in a hepatoma cell line. Science 199; 285:110-113.
22. Domitrovich A M, Felmlee D J, Siddiqui A. Hepatitis C Virus Nonstructural Proteins Inhibit Apolipoprotein B100 Secretion. The Journal of Biological Chemistry 2005; 280:39802-39808.
23. Deforges S, Evlashev A, Perret M, Sodoyer M, Pouzol S, Scoazec J Y, Bonnaud B, et al. Expression of hepatitis C virus proteins in epithelial intestinal cells in vivo. J Gen Virol 2004; 85(Pt 9):2515-2523.
24. Nahmias Y, Casali M, Barbe L, Berthiaume F, Yarmush M L. Liver endothelial cells promote LDL-R expression and the uptake of HCV-like particles in primary rat and human hepatocytes. Hepatology 2006; 43:257-265.
25. Agnello V, Abel G, Elfahal M, Knight G B, Zhang Q X. Hepatitis C virus and other flaviviridae viruses enter cells via low density lipoprotein receptor. PNAS 1999; 96:12766-12771.
26. Maillard P, Huby T, Andréo U, Moreau M, Chapman J, Budkowska A. The interaction of natural hepatitis C virus with human scavenger receptor SR-BI/Cla1 is mediated by ApoB containing lipoproteins. FASEB J. 2006; 20:735-737.
27. Barth H, Schnober E K, Zhang F, Linhardt R J, Depla E, Boson B, Cosset F L, et al. Viral and cellular determinants of the hepatitis C virus envelope-heparan sulfate interaction. Journal of Virology 2006; 80:10579-10590.
28. Kanno S-i, Tomizawa A, Hiura T, Osanai Y, Shouji A, Ujibe M, Ohtake T, et al. Inhibitory Effects of Naringenin on Tumor Growth in Human Cancer Cell Lines and Sarcoma 5-180-Implanted Mice. Biol Pharm Bull. 2005; 28:527-530.
29. Moon Y J, Wang X, Morris M E. Dietary flavonoids: effects on xenobiotic and carcinogen metabolism. Toxicol In Vitro. 2006; 20:187-210.
30. Huong D T, Takahashi Y, Ide T. Activity and mRNA levels of enzymes involved in hepatic fatty acid oxidation in mice fed citrus flavonoids. Nutrition 2006; 22:546-552.
31. Jung U J, Kim H J, Lee J S, Lee M K, Kim H O, Park E J, Kim H K, et al. Naringin supplementation lowers plasma lipids and enhances erythrocyte antioxidant enzyme activities in hypercholesterolemic subjects. Clinical Nutrition 2003; 22:561-568.
32. Lee C-H, Jeong T-S, Choi Y-K, Hyun B-H, Oh G-T, Kim E-H, Kim J-R, et al. Anti-atherogenic effect of citrus flavonoids, naringin and naringenin, associated with hepatic ACAT and aortic VCAM-1 and MCP-1 in high cholesterol-fed rabbits. Biochem Biophys Res Commun 2001; 284:681-688.
33. Kim S-Y, Kim H-J, Lee M-K, Jeon S-M, Do G-M, Kwon E-Y, Cho Y-Y, et al. Naringin time-dependently lowers hepatic cholesterol biosynthesis and plasma cholesterol in rats fed high-fat and high-cholesterol diet. J Med Food 2006; 9:582-586.
34. EKMMA8 Eksperimentalna Meditsina i Morfologiya. In. Volume 19. Sofia, Bulgaria, 1980; 207.
35. Mercer D F, Schiller D E, Elliott J F, Douglas D N, Hao C, Rinfret A, Addison W R, et al. Hepatitis C virus replication in mice with chimeric human livers. Nature Medicine 2001; 7:927-933.
36. Nahmias, Y. et al. Apolipoprotein B dependent Hepatitis C Virus Secretion is Inhibited by the Grapefruit Flavonoid Naringenin Hepatology, Epub Jan. 8, 2008.
37. Kanaze, F. I., Bounartzi, M. I., Georgarakis, M. & Niopas, I. Pharmacokinetics of the citrus flavanone aglycones hesperetin and naringenin after single oral administration in human subjects. European Journal of Clinical Nutrition 61, 472-477 (2007).
38. Miyake, K. et al. Improvement of Solubility and Oral Bioavailability of Rutin by Complexation with 2-Hydroxypropyl-b-cyclodextrin. Pharmaceutical Development and Technology 5, 399-407 (2000).
39. Gao, J., Hugger, E. D., Beck-Westermeyer, M. S. & Borchardt, R. T. in Current Protocols in Pharmacology (2000) (eds. S. J. Enna & M. Williams) 7.2.1-7.2.23 (John Wiley & Sons, 2000).
40. Guidotti L G, Chisari F V. Immunobiology and Pathogenesis of Viral Hepatitis. Annual Review of Pathology: Mechanisms of Disease 2006; 1:23-61.
41. Kapadia S B, Chisari F V. Hepatitis C virus RNA replication is regulated by host geranylgeranylation and fatty acids. PNAS 2005; 102:2561-2566.
42. Huang H, Sun F, Owen D M, Li W, Chen Y, Gale M, Jr., Ye J. Hepatitis C virus production by human hepatocytes dependent on assembly and secretion of very low-density lipoproteins. Proc Natl Acad Sci USA 2007; 104:5848-5853.
43. Nahmias Y, Goldwasser J, Casali M, Poll D v, Wakita T, Chung R T, Yarmush M L. Apolipoprotein B dependent Hepatitis C Virus Secretion is Inhibited by the Grapefruit Flavonoid Naringenin Hepatology 2008:Epub January 8.
44. Fraser C S, Doudna J A. Structural and mechanistic insights into hepatitis C viral translation initiation. Nat Rev Microbiol 2007; 5:29-38.
45. Penin F, Dubuisson J, Rey F A, Moradpour D, Pawlotsky J-M. Structural Biology of Hepatitis C Virus. Hepatology 2004; 39:5-19.
46. Quinkert D, Bartenschlager R, Lohmann V. Quantitative analysis of the hepatitis C virus replication complex. Journal of Virology 2005; 79:13594-13605.
47. Kashiwagi T, Hara K, Kohara M, Iwahashi J, Hamada N, Honda-Yoshino H, Toyoda T. Promoter/origin structure of the complementary strand of hepatitis C virus genome. Journal of Biological Chemistry 2002; 277:28700-28705.
48. Gastaminza P, Kapadia S B, Chisari F V. Differential Biophysical Properties of Infectious Intracellular and Secreted Hepatitis C Virus Particles. Journal of Virology 2006; 80:11074-11081.
49. Loo Y-M, Owen D M, Li K, Erickson A K, Johnson C L, Fish P M, Carney S, et al. Viral and therapeutic control of IFN-beta promoter stimulator 1 during hepatitis C virus infection. Proc Natl Acad Sci USA 2006; 103:6001-6006.
50. Pekow J R, Bhan A K, Zheng H, Chung R T. Hepatic steatosis is associated with increased frequency of hepatocellular carcinoma in patients with hepatitis C-related cirrhosis. Cancer 2007; [Epub ahead of print].
51. Sabile A, Perlemuter G, Bono F, Kohara K, Demaugre F, Kohara M, Matsuura Y, et al. Hepatitis C virus core protein binds to apolipoprotein AII and its secretion is modulated by fibrates. Hepatology 1999; 30:1064-1076.
52. Barba G, Harper F, Harada T, Kohara M, Goulinet S, Matsuura Y, Eder G, et al. Hepatitis C virus core protein shows a cytoplasmic localization and associates to cellular lipid storage droplets. PNAS 1997; 94:1200-1205.
52. Andre P, Perlemuter G, Budkowska A, Bre'chot C, Lotteau V. Hepatitis C Virus Particles and Lipoprotein Metabolism. Semin Liver Dis. 2005; 25:93-104.
54. Lohmann V, Korner F, Koch J, Herian U, Theilmann L, Bartenschlager R. Replication of subgenomic hepatitis C virus RNAs in a hepatoma cell line. Science 199; 285:110-113.
55. Kim S S, Peng L F, Lin W, Choe W-H, Sakamoto N, Schreiber S L, Chung R T. A Cell-Based, High-Throughput Screen for Small Molecule Regulators of Hepatitis C Virus Replication. Gastroenterology 2007; 132:311-320.
56. Gastaminza P, Cheng G, Wieland S, Zhong J, Liao W, Chisari F V. Cellular Determinants of Hepatitis C Virus Assembly, Maturation, Degradation, and Secretion. Journal of Virology 2008; 82:2120-2129.
57. Thomssen R, Bonk S, Propfe C, Heermann K H, Kochel H G, Uy A. Association of hepatitis C virus in human sera with beta-lipoprotein. Med Microbiol Immunol. 1992; 181:293-300.
58. Nahmias Y, Casali M, Barbe L, Berthiaume F, Yarmush M L. Liver endothelial cells promote LDL-R expression and the uptake of HCV-like particles in primary rat and human hepatocytes. Hepatology 2006; 43:257-265.
59. Agnello V, Abel G, Elfahal M, Knight G B, Zhang Q X. Hepatitis C virus and other flaviviridae viruses enter cells via low density lipoprotein receptor. PNAS 1999; 96:12766-12771.
60. Maillard P, Huby T, Andreo U, Moreau M, Chapman J, Budkowska A. The interaction of natural hepatitis C virus with human scavenger receptor SR-BI/Cla1 is mediated by ApoB-containing lipoproteins. FASEB J. 2006; 20::735-737.
61. Barth H, Schnober E K, Zhang F, Linhardt R J, Depla E, Boson B, Cosset F L, et al. Viral and cellular determinants of the hepatitis C virus envelope-heparan sulfate interaction. Journal of Virology 2006; 80:10579-10590.
62. Kanno S-i, Tomizawa A, Hiura T, Osanai Y, Shouji A, Ujibe M, Ohtake T, et al. Inhibitory Effects of Naringenin on Tumor Growth in Human Cancer Cell Lines and Sarcoma S-180-Implanted Mice. Biol Pharm Bull. 2005; 28:527-530.
63. Wilcox L J, Borradaile N M, Dreu LEd, Huff M W. Secretion of hepatocyte apoB is inhibited by the flavonoids, naringenin and hesperetin, via reduced activity and expression of ACAT2 and MTP. Journal of Lipid Research 2001; 42:725-734.
64. Moon Y J, Wang X, Morris M E. Dietary flavonoids: effects on xenobiotic and carcinogen metabolism. Toxicol In Vitro. 2006; 20:187-210.
65. Huong D T, Takahashi Y, Ide T. Activity and mRNA levels of enzymes involved in hepatic fatty acid oxidation in mice fed citrus flavonoids. Nutrition 2006; 22:546-552.
66. Kurowska E, Borradaile N, Spence J D, Carroll K K. Hypocholesterolemic effects of dietary citrus juices in rabbits. Nutr. Res. 2000; 20:121-129.
67. Allister E M, Borradaile N M, Edwards J Y, Huff M W. Inhibition of microsomal triglyceride transfer protein expression and apolipoprotein B100 secretion by the citrus flavonoid naringenin and by insulin involves activation of the mitogen-activated protein kinase pathway in hepatocytes. Diabetes 2005; 54:1676-1683.
68. Jung U J, Kim H J, Lee J S, Lee M K, Kim H O, Park E J, Kim H K, et al. Naringin supplementation lowers plasma lipids and enhances erythrocyte antioxidant enzyme activities in hypercholesterolemic subjects. Clinical Nutrition 2003; 22:561-568.
69. Lee C-H, Jeong T-S, Choi Y-K, Hyun B-H, Oh G-T, Kim E-H, Kim J-R, et al. Anti-atherogenic effect of citrus flavonoids, naringin and naringenin, associated with hepatic ACAT and aortic VCAM-1 and MCP-1 in high cholesterol-fed rabbits. Biochem Biophys Res Commun 2001; 284:681-688.
70. Kim S-Y, Kim H-J, Lee M-K, Jeon S-M, Do G-M, Kwon E-Y, Cho Y-Y, et al. Naringin time-dependently lowers hepatic cholesterol biosynthesis and plasma cholesterol in rats fed high-fat and high-cholesterol diet. J Med Food 2006; 9:582-586.
71. Mitchell A E, Burns S A, Rudolf J L. Isozyme- and gender-specific induction of glutathione S-transferases by flavonoids. Arch Toxicol. 2007; [Epub ahead of print].
72. Jeon S M, Kim H K, Kim H J, Do G M, Jeong T S, Park Y B, Choi M S. Hypocholesterolemic and antioxidative effects of naringenin and its two metabolites in high-cholesterol fed rats. Transl Res. 2007; 149:15-21.
73. Kurowska E M, Manthey J A, Casaschi A, Theriault A G. Modulation of HepG2 Cell Net Apolipoprotein B Secretion by the Citrus Polymethoxyflavone, Tangeretin. Lipids 2004; 39:143-151.
74. Borradaile N M, Dreu LEd, Barrett P H R, Huff M W. Inhibition of hepatocyte apoB secretion by naringenin: enhanced rapid intracellular degradation independent of reduced microsomal cholesteryl esters. Journal of Lipid Research 2002; 43.
75. Borradaile N M, Dreu LEd, Huff M W. Inhibition of Net HepG2 Cell Apolipoprotein B Secretion by the Citrus Flavonoid Naringenin Involves Activation of Phosphatidylinositol 3-Kinase, Independent of Insulin Receptor Substrate-1 Phosphorylation. Diabetes 2003; 52:2554-2561.
76. Kaul D, Sikand K, Shukla A R. Effect of Green Tea Polyphenols on the Genes with Atherosclerotic Potential. Phytother. Res. 2004; 18:177-179.
77. Xiao C W, Mei J, Wood C M. Effect of soy proteins and isoflavones on lipid metabolism and involved gene expression. Frontiers in Bioscience 2008; 13:2660-2673.
78. Crozier A, Jaganath I B, Clifford M N. Dietary phenolics: chemistry, bioavailability and effects on health. Natural product reports 2009; 26:1001-1043.
79. García-Lafuente A, Guillamón E, Villares A, Rostagno M A, Martinez J A. Flavonoids as anti-inflammatory agents: implications in cancer and cardiovascular disease. Inflamm Res 2009; 58:537-552.
80. Carluccio M A, Siculella L, Ancora M A, Massaro M, Scoditti E, Storelli C, Visioli F, et al. Olive oil and red wine antioxidant polyphenols inhibit endothelial activation: antiatherogenic properties of Mediterranean diet phytochemicals. Arteriosclerosis, Thrombosis, and Vascular Biology 2003; 23:622-629.
81. Kaul D, Shukla A R, Sikand K, Dhawan V. Effect of herbal polyphenols on atherogenic transcriptome. Mol Cell Biochem 2005; 278:177-184.
82. Sun B, Spranger I, Yang J, Leandro C, Guo L, Canário S, Zhao Y, et al. Red wine phenolic complexes and their in vitro antioxidant activity. J Agric Food Chem 2009; 57:8623-8627.
83. Wang Y, Ho C T. Polyphenolic chemistry of tea and coffee: a century of progress. J Agric Food Chem 2009; 57:8109-8114.
84. Mulvihill E E, Allister E M, Sutherland B G, Telford D E, Sawyez C G, Edwards J Y, Markle J M, et al. Naringenin prevents dyslipidemia, apoB overproduction and hyperinsulinemia in LDL-receptor null mice with diet-induced insulin resistance. Diabetes 2009.
85. Choe S C, Kim H S, Jeong T S, Bok S H, Park Y B. Naringin has an antiatherogenic effect with the inhibition of intercellular adhesion molecule-1 in hypercholesterolemic rabbits. J Cardiovasc Pharmacol 2001; 38:947-955.
86. Wilcox, Borradaile, Huff. Antiatherogenic Properties of Naringenin, a Citrus Flavonoid. Cardiovascular Drug Reviews 1999.
87. Renugadevi J, Prabu S M. Naringenin protects against cadmium-induced oxidative renal dysfunction in rats. Toxicology 2009; 256:128-134.
88. Wilcox, Borradaile, Dreu d, Huff. Secretion of hepatocyte apoB is inhibited by the flavonoids, naringenin and hesperetin, via reduced activity and expression of ACAT2 and MTP. J Lipid Res 2001; 42:725-734.
89. Allister, Borradaile, Edwards, Huff. Inhibition of microsomal triglyceride transfer protein expression and apolipoprotein B100 secretion by the citrus flavonoid naringenin and by insulin involves activation of the mitogen-activated protein kinase pathway in hepatocytes. Diabetes 2005; 54:1676-1683.
90. Moon Y J, Wang X, Morris M E. Dietary flavonoids: effects on xenobiotic and carcinogen metabolism. Toxicol In Vitro 2006; 20:187-210.
91. Jeon S M, Kim H K, Kim H J, Do G M, Jeong T S, Park Y B, Choi M S. Hypocholesterolemic and antioxidative effects of naringenin and its two metabolites in high-cholesterol fed rats. Transl Res 2007; 149:15-21.
92. Jung U J, Kim H J, Lee J S, Lee M K, Kim H O, Park E J, Kim H K, et al. Naringin supplementation lowers plasma lipids and enhances erythrocyte antioxidant enzyme activities in hypercholesterolemic subjects. Clin Nutr 2003; 22:561-568.
93. Kurowska E M, Borradaile, Spence J D. Hypocholesterolemic effects of dietary citrus juices in rabbits. Nutrition Research 2000.
94. Nahmias, Goldwasser, Casali, Poll v, Wakita, Chung, Yarmush M. Apolipoprotein B-dependent hepatitis C virus secretion is inhibited by the grapefruit flavonoid naringenin. Hepatology 2008; 47:1437-1445.
95. Rajewski, Stella. Pharmaceutical applications of cyclodextrins. 2. In vivo drug delivery. Journal of pharmaceutical sciences 1996; 85:1142-1169.
96. Stella, Rajewski. Cyclodextrins: their future in drug formulation and delivery. Pharm Res 1997; 14:556-567.
97. Miyake, Arima, Hirayama, Yamamoto, Horikawa, Sumiyoshi, Noda, et al. Improvement of solubility and oral bioavailability of rutin by complexation with 2-hydroxypropyl-beta-cyclodextrin. Pharmaceutical development and technology 2000; 5:399-407.
98. Gao J, Hugger E D, Beck-Westermeyer M S, Borchardt R T: Estimating Intestinal Mucosal Permeation of Compounds Using Caco-2 Cell Monolayers. In: Enna S J, Williams M, eds. Current Protocols in Pharmacology, 2000.
99. Tommasini, S., et al., Improvement in solubility and dissolution rate of flavonoids by complexation with beta-cyclodextrin. Journal of pharmaceutical and biomedical analysis 2004; 35:379-387.
100. Stella V J, He Q. Cyclodextrins. Toxicol Pathol 2008; 36:30-42.
101. Tommasini, S., et al., Combined effect of pH and polysorbates with cyclodextrins on solubilization of naringenin. Journal of pharmaceutical and biomedical analysis, 2004; 36:327-333.
102. Ficarra, R., et al., Study of flvonoids/β-cyclodextrins inclusion complexes by NMR, FT-RI, DSC, X-ray investigation. Journal of pharmaceutical and biomedical analysis, 2002; 29:1005-1014.

All references cited herein are hereby incorporated by reference in their entirety.

The present invention can be defined in any of the following numbered paragraphs:
1. A method of treating a viral infection comprising:
   selecting a patient in need of treatment for viral infection;
   administering to the patient an effective amount of a flavonoid-sugar complex.
2. The method of paragraph 1, wherein administering is orally administering to the patient in oral dosage form.
3. The method of paragraph 1, wherein the viral infection is a hepatitis C virus infection.
4. The method of paragraph 1, wherein the sugar is hydrpxypropyl-β-cyclodextrin.
5. The method of paragraph 1, wherein flavonoid is naringenin.
6. The method of paragraph 1 further comprising a pharmaceutically acceptable carrier.
7. The method of paragraph 1, wherein the oral dosage form is a tablet.
8. The method of paragraph 7, wherein the tablet is a controlled release tablet.
9. The method of paragraph 1, wherein the administering step is at least 1 hour before the patient's next intake of food.
10. The method of paragraph 2, wherein the oral dosage form contains from 1 to 5000 mg/dose naringenin.
11. A method of treating inflammation comprising:
   selecting a patient in need of treatment for inflammation;
   administering to the patient an effective amount of a flavonoid-sugar complex.
12. The method of paragraph 11, wherein the administering is orally administering to the patient an oral dosage form.
13. The method of paragraph 11, wherein the sugar is hydrpxypropyl-β-cyclodextrin.
14. The method of paragraph 11, wherein flavonoid is naringenin.
15. The method of paragraph 11 further comprising a pharmaceutically acceptable carrier.
16. The method of paragraph 11, wherein the oral dosage form is a tablet.
17. The method of paragraph 16, wherein the tablet is a controlled release tablet.
18. The method of paragraph 11, wherein the administering step is at least 1 hour before the patient's next intake of food.
19. The method of paragraph 12, wherein the oral dosage form contains from 1 to 5000 mg/dose naringenin.
20. A method of treating dyslipidemia comprising:
   selecting a patient in need of treatment for dyslipidemia;
   administering to the patient an effective amount of a flavonoid-sugar complex.
21. The method of paragraph 20, wherein the administering is orally administering to the patient an oral dosage form.
22. The method of paragraph 20, wherein the sugar is hydrpxypropyl-β-cyclodextrin.
23. The method of paragraph 20, wherein flavonoid is naringenin.
24. The method of paragraph 20, further comprising a pharmaceutically acceptable carrier.
25. The method of paragraph 20, wherein the oral dosage form is a tablet.
26. The method of paragraph 25, wherein the tablet is a controlled release tablet.
27. The method of paragraph 20, wherein the administering step is at least 1 hour before the patient's next intake of food.
28. The method of paragraph 21, wherein the oral dosage form contains from 1 to 5000 mg/dose naringenin.
29. A method of treating insulin resistance or diabetes comprising:
   selecting a patient in need of treatment for insulin resistance or diabetes;
   administering to the patient an effective amount of a flavonoid-sugar complex.
30. The method of paragraph 29, wherein the administering is orally administering to the patient an oral dosage form.
31. The method of paragraph 29, wherein the sugar is hydrpxypropyl-β-cyclodextrin.
32. The method of paragraph 29, wherein flavonoid is naringenin.
33. The method of paragraph 29 further comprising a pharmaceutically acceptable carrier.
34. The method of paragraph 29, wherein the oral dosage form is a tablet.
35. The method of paragraph 34, wherein the tablet is a controlled release tablet.
36. The method of paragraph 29, wherein the administering step is at least 1 hour before the patient's next intake of food.
37. The method of paragraph 30, wherein the oral dosage form contains from 1 to 5000 mg/dose naringenin.
38. A pharmaceutical composition comprising a flavonoid-sugar complex.
39. The composition of paragraph 38, wherein the sugar is a cyclodextrin.
40. The composition of paragraph 39, wherein the cyclodextrin is β-cyclodextrin.
41. The composition of paragraph 38, wherein the sugar is hydrpxypropyl-β-cyclodextrin.
42. The composition of paragraph 38, wherein flavonoid is naringenin.
43. The composition of paragraph 38 further comprising a pharmaceutically acceptable carrier.
44. A pharmaceutical composition consisting essentially of a naringenin β-cyclodextrin complex.
45. The composition of paragraphs 38-45, wherein the composition is a tablet.
46. A method of increasing the bioavailability of a pharmaceutically active compound comprising: selecting a patient in need of treatment with the pharmaceutically active; administering to the subject the pharmaceutically active compound and a flavonoid-sugar complex, wherein the pharmaceutically active compound is metabolized by Cytochrome P450.
47. The method of paragraph 46, wherein the pharmaceutically active compound is a HMG-CoA reductase inhibitor.
48. The method of paragraph 47, wherein the HMG-CoA reductase inhibitor is selected from the group consisting of atrovastatin, cerivastatin. Fluvastatin, lovastatin, mevastatin, pitavastin, praystatin, rosuvastatin, simvastatin, and combinations thereof.
49. The method of paragraph 46, wherein the flavonoid-sugar complex is administrated orally to the patient in an oral dosage form.
50. The method of paragraph 49, wherein the oral dosage form contains from 1 to 5000 mg/dose naringenin.
51. The method of paragraph 49, wherein the oral dosage form is a tablet.
52. The method of paragraph 51, wherein the tablet is a controlled release tablet.
53. The method of paragraph 46, wherein the sugar is hydrpxypropyl-β-cyclodextrin.
54. The method of paragraph 46, wherein flavonoid is naringenin.

1-45. (canceled)

46. A method of increasing the bioavailability of a pharmaceutically active compound comprising: selecting a patient in need of treatment with the pharmaceutically active;
administering to the subject the pharmaceutically active compound and a flavonoid-sugar complex, wherein the pharmaceutically active compound is metabolized by Cytochrome P450.

47. The method of claim 46, wherein the pharmaceutically active compound is a HMG-CoA reductase inhibitor.

48. The method of claim 47, wherein the HMG-CoA reductase inhibitor is selected from the group consisting of atrovastatin, cerivastatin. Fluvastatin, lovastatin, mevastatin, pitavastin, praystatin, rosuvastatin, simvastatin, and combinations thereof.

49. The method of claim 46, wherein the flavonoid-sugar complex is administrated orally to the patient in an oral dosage form.

50. The method of claim 49, wherein the oral dosage form contains from 1 to 5000 mg/dose naringenin.

51. The method of claim 49, wherein the oral dosage form is a tablet.

52. The method of claim 51, wherein the tablet is a controlled release tablet.

53. The method of claim 46, wherein the sugar is hydrpxypropyl-β-cyclodextrin.

54. The method of claim 46, wherein flavonoid is naringenin.
